(19) United States
(12) Patent Application Publication
(10) Pub. No.: US 2026/0217848 A1
(43) Pub. Date: Jul. 30, 2026

(54) EPITOPE EDITING AND MULTI-SPECIFIC IMMUNCE-CELL ENGAGER THERAPY AND METHODS OF USE

(71) Applicants: The Wistar Institute of Anatomy and Biology, Philadelphia, PA (US); The Trustees of the University of Pennsylvania, Philadelphia, PA (US)

(72) Inventors: Ryan O'Connell, Philadelphia, PA (US); David Weiner, Merion, PA (US); Nils Welhausen, Philadelphia, PA (US); Carl June, Philadelphia, PA (US); Saar Gill, Philadelphia, PA (US)

(21) Appl. No.: 19/159,102

(22) PCT Filed: Feb. 26, 2024
(86) PCT No.: PCT/US2024/017249

§ 371 Date: Aug. 22, 2025

Related U.S. Application Data
(60) Provisional application No. 63/486,751, filed on Feb. 24, 2023.
(60) Provisional application No. 63/503,008, filed on May 18, 2023.

(51) Int. Cl. C07K 16/28; A61K 39/00; A61K 39/395; A61K 40/11; A61K 40/31; A61K 40/33; A61K 40/42; A61P 35/02

(52) U.S. Cl. CPC C07K 16/289; A61K 39/3955; A61K 40/11; A61K 40/31; A61K 40/33; A61K 40/4224; A61P 35/02; C07K 16/2809; A61K 2039/505; C07K 2317/24; C07K 2317/31; C07K 2317/35; C07K 2317/73

(57) ABSTRACT

Disclosed herein are compositions comprising anti-CD45 bispecific T cell engagers as well as variants thereof, combinations thereof, and methods of use thereof for the treatment of cancer, as well as methods of improved immunotherapy through the combined use of multi-specific antibodies and epitope editing.

### CROSS REFERENCE TO RELATED APPLICATIONS

This application claims priority to U.S. Provisional Application No. 63/486,751, filed Feb. 24, 2023, and U.S. Provisional Application No. 63/503,008, filed May 18, 2023, each of which is hereby incorporated by reference herein in its entirety.

### BACKGROUND OF THE INVENTION

In the absence of cell-surface cancer-specific antigens, immunotherapies such as chimeric antigen receptor (CAR) T cells, monoclonal antibodies or bispecific engagers must target lineage antigens. Currently, such immunotherapies are individually designed and tested for each disease. This approach is inefficient and limited to a few lineage antigens for which the on-target/off-tumor toxicities are clinically tolerated.

Thus there is need in the art for therapeutic methods that leverage normally expressed proteins for antibody targeting, while diminishing off-target effects to healthy cells like the effector cells. The current invention satisfies this need.

### SUMMARY OF THE INVENTION

In one embodiment, the invention relates to a synthetic multi-specific immune cell engager (ICE), wherein the synthetic multi-specific immune cell engager comprises at least one CD45 antigen binding domain, and at least one immune cell engaging domain.

In one embodiment, the synthetic multi-specific immune cell engager comprises at least two CD45 antigen binding domains, and at least one immune cell engaging domain.

In one embodiment, the multi-specific ICE comprises at least one antigen binding domain comprising a variable heavy chain amino acid sequence having at least about 90% identity over the entire length of the amino acid sequence to SEQ ID NO:1 comprising at least the CDR sequences of SEQ ID NO: 1; or a fragment of SEQ ID NO:1 comprising at least the CDR sequences of SEQ ID NO:1; and a variable light chain amino acid sequence an amino acid sequence having at least about 90% identity over the entire length of the amino acid sequence to SEQ ID NO:2 comprising at least the CDR sequences of SEQ ID NO:2; or a fragment of SEQ ID NO:2 comprising at least the CDR sequences of SEQ ID NO:2.

In one embodiment, the multi-specific ICE comprises at least one antigen binding domain comprising an amino acid sequence having at least about 90% identity over the entire length of the amino acid sequence to SEQ ID NO:5 or SEQ ID NO:7, wherein the amino acid sequence comprising at least the CDR sequences of SEQ ID NO:5 or SEQ ID NO:7; or a fragment of SEQ ID NO:5 or SEQ ID NO:7 comprising at least the CDR sequences of SEQ ID NO:5 or SEQ ID NO: 7.

In one embodiment, the immune cell is a T cell or a naturally killer (NK) cell. In one embodiment, the immune cell engaging domain targets CD3, the T cell receptor (TCR), CD28, CD16, NKG2D, Ox40, 4-1BB, CD2, CD5, CD40, FcgRs, FceRs, FcaRs or CD95.

In one embodiment, the immune cell engaging domain targets CD3.

In one embodiment, the synthetic multi-specific ICE comprises an antigen binding domain comprising a variable heavy chain amino acid sequence having at least about 90% identity over the entire length of the amino acid sequence to SEQ ID NO:3 comprising at least the CDR sequences of SEQ ID NO:3; or a fragment of SEQ ID NO:3 comprising at least the CDR sequences of SEQ ID NO:3; and a variable light chain amino acid sequence an amino acid sequence having at least about 90% identity over the entire length of the amino acid sequence to SEQ ID NO:4 comprising at least the CDR sequences of SEQ ID NO:4; or a fragment of SEQ ID NO:4 comprising at least the CDR sequences of SEQ ID NO:4.

In one embodiment, the synthetic multi-specific ICE comprises an amino acid sequence having at least about 90% identity over the entire length of the amino acid sequence to SEQ ID NO:9, wherein the amino acid sequence comprising at least the CDR sequences of SEQ ID NO:9; or a fragment of SEQ ID NO:9 comprising at least the CDR sequences of SEQ ID NO:9.

In one embodiment, the synthetic multi-specific ICE of claim 1 comprising an amino acid sequence having at least about 90% identity over the entire length of the amino acid sequence to SEQ ID NO:11, SEQ ID NO: 12, SEQ ID NO:13. SEQ ID NO:14, SEQ ID NO:19, SEQ ID NO:20, SEQ ID NO:21, SEQ ID NO:22, SEQ ID NO:23, SEQ ID NO:24. SEQ ID NO:25, SEQ ID NO:26, SEQ ID NO:27 or SEQ ID NO:28, wherein the amino acid sequence comprising at least the CDR sequences of SEQ ID NO: 11, SEQ ID NO:12, SEQ ID NO:13, SEQ ID NO:14, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28; or a fragment of SEQ ID NO: 11, SEQ ID NO:12, SEQ ID NO: 13. SEQ ID NO:14, SEQ ID NO: 19, SEQ ID NO:20, SEQ ID NO:21, SEQ ID NO:22. SEQ ID NO:23, SEQ ID NO:24, SEQ ID NO:25, SEQ ID NO:26, SEQ ID NO:27 or SEQ ID NO:28 comprising at least the CDR sequences of SEQ ID NO:11, SEQ ID NO:12, SEQ ID NO:13, SEQ ID NO:14, SEQ ID NO: 19, SEQ ID NO:20, SEQ ID NO:21, SEQ ID NO:22. SEQ ID NO:23, SEQ ID NO:24, SEQ ID NO:25, SEQ ID NO:26, SEQ ID NO:27 or SEQ ID NO:28.

In one embodiment, the invention relates to a nucleic acid molecule encoding a synthetic multi-specific ICE, wherein the synthetic multi-specific immune cell engager comprises at least one CD45 antigen binding domain, and at least one immune cell engaging domain.

In one embodiment, the nucleic acid molecule is selected from the group consisting of an RNA molecule and a DNA molecule.

In one embodiment, the nucleic acid molecule comprises a nucleotide sequence having at least about 90% identity over an entire length of the nucleic acid sequence to SEQ ID NO:6, SEQ ID NO:8, or SEQ ID NO:10.

In one embodiment, the nucleic acid molecule comprises a fragment of a nucleotide sequence having at least about 90% identity over at least 65% of the nucleic acid sequence to SEQ ID NO:6, SEQ ID NO:8, or SEQ ID NO:10.

In one embodiment, the nucleic acid molecule comprises a nucleotide sequence of SEQ ID NO:6, SEQ ID NO:8, or SEQ ID NO:10.

In one embodiment, the nucleic acid molecule comprises a fragment of a nucleotide sequence comprising at least 65% of a nucleotide sequence of SEQ ID NO:6, SEQ ID NO:8, or SEQ ID NO: 10.

In one embodiment, the nucleic acid molecule comprises a nucleotide of SEQ ID NO: 15. SEQ ID NO:16, SEQ ID NO: 17 or SEQ ID NO:18, or a fragment or variant thereof.

In one embodiment, the nucleic acid molecule comprises an expression vector.

In one embodiment, the invention relates to a composition comprising a synthetic multi-specific ICE, wherein the synthetic multi-specific immune cell engager comprises at least one CD45 antigen binding domain, and at least one immune cell engaging domain. In one embodiment, the composition further comprises a pharmaceutically acceptable excipient. In one embodiment, the composition further comprises at least one immune checkpoint inhibitor or a nucleic acid molecule encoding an immune checkpoint inhibitor. In one embodiment, the checkpoint inhibitor is an inhibitor of PD-1, an inhibitor of PD-L-1, an inhibitor of cytotoxic T-lymphocyte antigen-4 (CTLA-4), an inhibitor of mucin-domain containing-3 (TIM-3), or an inhibitor of Lymphocyte Activating 3 (LAG3).

In one embodiment, the invention relates to a composition comprising a nucleic acid molecule encoding a synthetic multi-specific ICE, wherein the synthetic multi-specific immune cell engager comprises at least one CD45 antigen binding domain, and at least one immune cell engaging domain. In one embodiment, the composition further comprises a pharmaceutically acceptable excipient. In one embodiment, the composition further comprises at least one immune checkpoint inhibitor or a nucleic acid molecule encoding an immune checkpoint inhibitor. In one embodiment, the checkpoint inhibitor is an inhibitor of PD-1, an inhibitor of PD-L-1, an inhibitor of cytotoxic T-lymphocyte antigen-4 (CTLA-4), an inhibitor of mucin-domain containing-3 (TIM-3), or an inhibitor of Lymphocyte Activating 3 (LAG3).

In one embodiment, the invention relates to a composition comprising lipid nanoparticle comprising a synthetic multi-specific ICE, wherein the synthetic multi-specific immune cell engager comprises at least one CD45 antigen binding domain, and at least one immune cell engaging domain. In one embodiment, the composition further comprises a pharmaceutically acceptable excipient. In one embodiment, the composition further comprises at least one immune checkpoint inhibitor or a nucleic acid molecule encoding an immune checkpoint inhibitor. In one embodiment, the checkpoint inhibitor is an inhibitor of PD-1, an inhibitor of PD-L-1, an inhibitor of cytotoxic T-lymphocyte antigen-4 (CTLA-4), an inhibitor of mucin-domain containing-3 (TIM-3), or an inhibitor of Lymphocyte Activating 3 (LAG3).

In one embodiment, the invention relates to a composition comprising lipid nanoparticle comprising a nucleic acid molecule encoding a synthetic multi-specific ICE, wherein the synthetic multi-specific immune cell engager comprises at least one CD45 antigen binding domain, and at least one immune cell engaging domain. In one embodiment, the composition further comprises a pharmaceutically acceptable excipient. In one embodiment, the composition further comprises at least one immune checkpoint inhibitor or a nucleic acid molecule encoding an immune checkpoint inhibitor. In one embodiment, the checkpoint inhibitor is an inhibitor of PD-1, an inhibitor of PD-L-1, an inhibitor of cytotoxic T-lymphocyte antigen-4 (CTLA-4), an inhibitor of mucin-domain containing-3 (TIM-3), or an inhibitor of Lymphocyte Activating 3 (LAG3).

In one embodiment, the invention relates to a method of preventing or treating CD45 expressing cancer in a subject, the method comprising administering to the subject a multi-specific ICE, wherein the synthetic multi-specific immune cell engager comprises at least one CD45 antigen binding domain, and at least one immune cell engaging domain, or a nucleic acid molecule encoding the multi-specific ICE, or a composition comprising a multi-specific ICE, wherein the synthetic multi-specific immune cell engager comprises at least one CD45 antigen binding domain, and at least one immune cell engaging domain or a nucleic acid molecule encoding the multi-specific ICE. In one embodiment, the CD45 expressing cancer is a blood cancer. In one embodiment, the blood cancer is selected from the group consisting of leukemia, myeloma, and lymphoma. In one embodiment, the blood cancer is acute myeloid leukemia (AML).

In one embodiment, the invention relates to a CD45-specific chimeric antigen receptor (CAR) molecule. In one embodiment, the CAR comprises an CD45 specific binding arm comprising a nucleotide sequence as set forth in SEQ ID NO:6 or SEQ ID NO:8.

In one embodiment, the invention relates to a composition comprising an CD45-specific chimeric antigen receptor (CAR) molecule. In one embodiment, the CAR comprises an CD45 specific binding arm comprising a nucleotide sequence as set forth in SEQ ID NO:6 or SEQ ID NO:8.

In one embodiment, the invention relates to a cell expressing a CD45-specific chimeric antigen receptor (CAR) molecule. In one embodiment, the CAR comprises an CD45 specific binding arm comprising a nucleotide sequence as set forth in SEQ ID NO:6 or SEQ ID NO:8. In one embodiment, the cell is an engineered T cell.

In one embodiment, the invention relates to a method of preventing or treating a disease or disorder in a subject in need thereof, the method comprising administering to the subject a multi-specific ICE wherein the synthetic multi-specific immune cell engager comprises at least one least one antigen binding domain, and at least one immune cell engaging domain, wherein the antigen binding domain is specific for a disease-associated antigen.

In one embodiment, the invention relates to a method of preventing or treating a disease or disorder in a subject in need thereof, the method comprising administering to the subject an engineered cell, wherein the engineered cell comprises a modification of at least one epitope that is bound by the multi-specific ICE. In one embodiment, the multi-specific antibody targets one or more of CD45, CD3, CD2, CD5, CD7, CD19, CD20, CD22, CD33, CCR5, CCR8, and BCMA. In one embodiment, the engineered cell comprises a modification in one or more of CD45, CD3, CD2, CD5, CD7, CD19, CD20, CD22, CD33, CCR5, CCR8, and BCMA to reduce binding of the multi-specific antibody to the engineered cell. In one embodiment, the engineered cell is a hematopoietic stem cell, T cell, B cell, NK cell, NK-T cell, neutrophil, macrophage, monocyte, dendritic cell, or any combination thereof. In one embodiment, the engineered cell is autologous or allogeneic with respect to the subject. In one embodiment, the engineered cell is epitope edited ex vivo prior to administration. In one embodiment, the disease or disorder is cancer, an autoimmune disease, or an infectious disease. In one embodiment, the disease or disorder is AML.

### BRIEF DESCRIPTION OF THE DRAWINGS

FIG. 1 depicts the antibody design and synthetic workflow.

FIG. 2 depicts data demonstrating that CD45-BiTEs express and display approximate MW.

FIG. 3 depicts data demonstrating size-exclusion chromatography purification of CD45-BiTEs.

FIG. 4 depicts data demonstrating that CD45-BiTEs bind T cell surface-expressed CD45 and CD3.

FIG. 5 depicts data demonstrating that CD45-BiTEs bind CD3- and CD45-expressing cells by flow cytometry.

FIG. 6 depicts data demonstrating that BC8-BiTEs are highly efficient at killing AML MOLM14 cells.

FIG. 7 depicts data demonstrating that anti-CD45/CD3 bispecific T cell engagers demonstrate high efficacy against AML cells while sparing epitope edited cells.

FIG. 8 depicts a diagram showing that the antibody can be monovalent for CD45 with 1:1 valency ratio for CD45 and CD3 (top) or bivalent for CD45, with two CD45 binding domains (bottom).

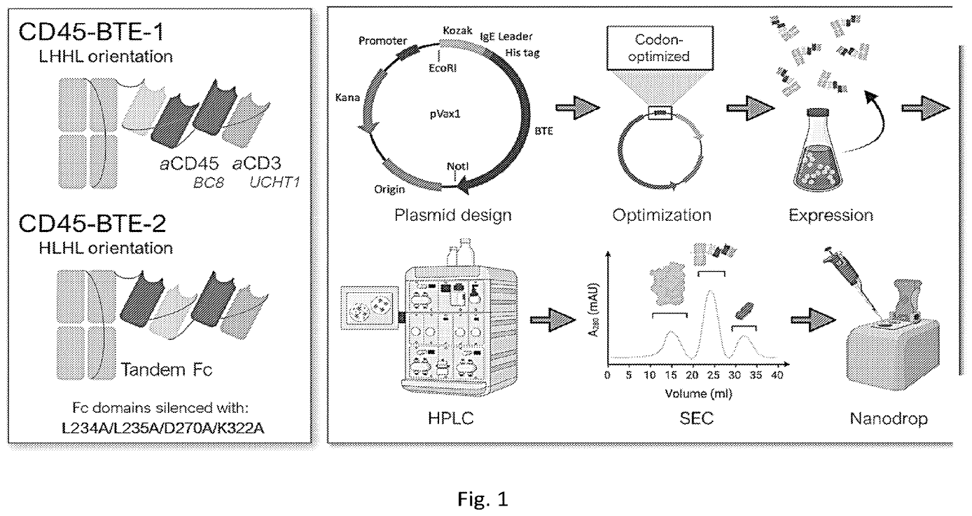

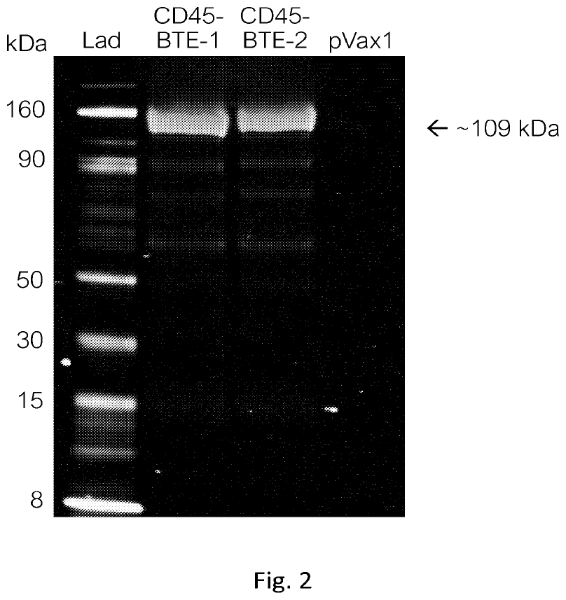

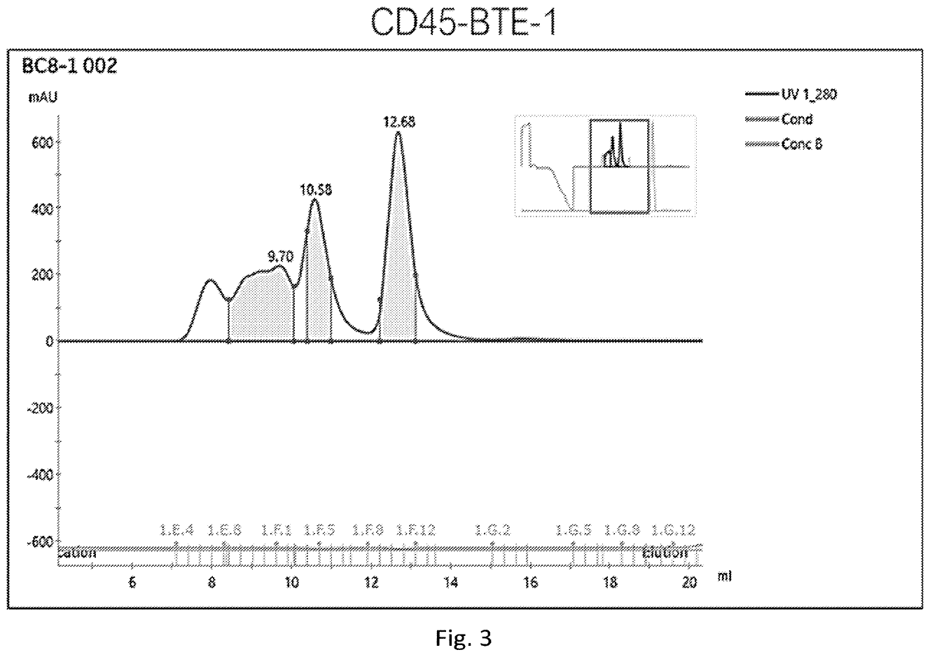

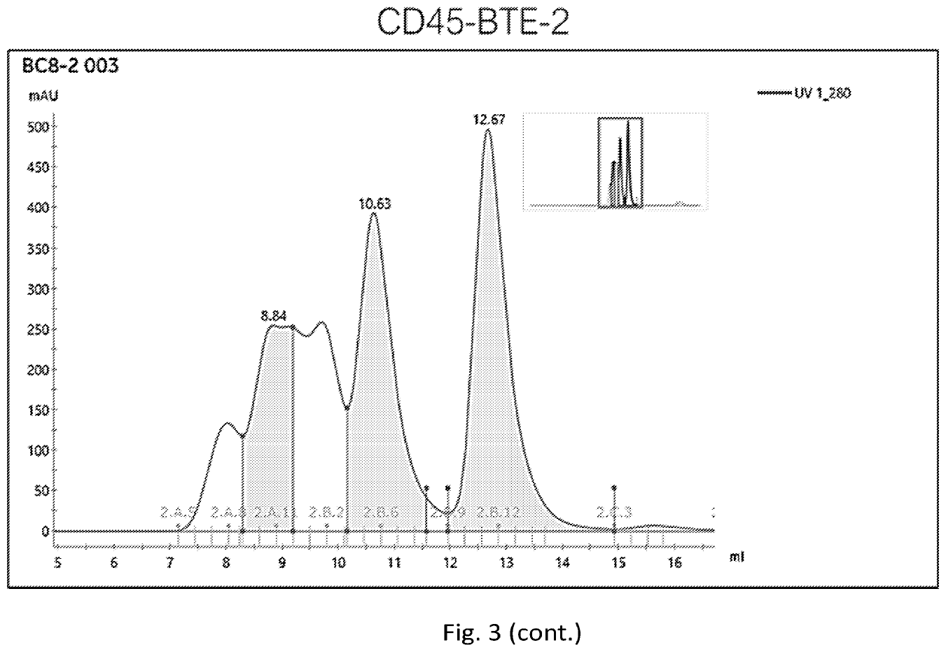

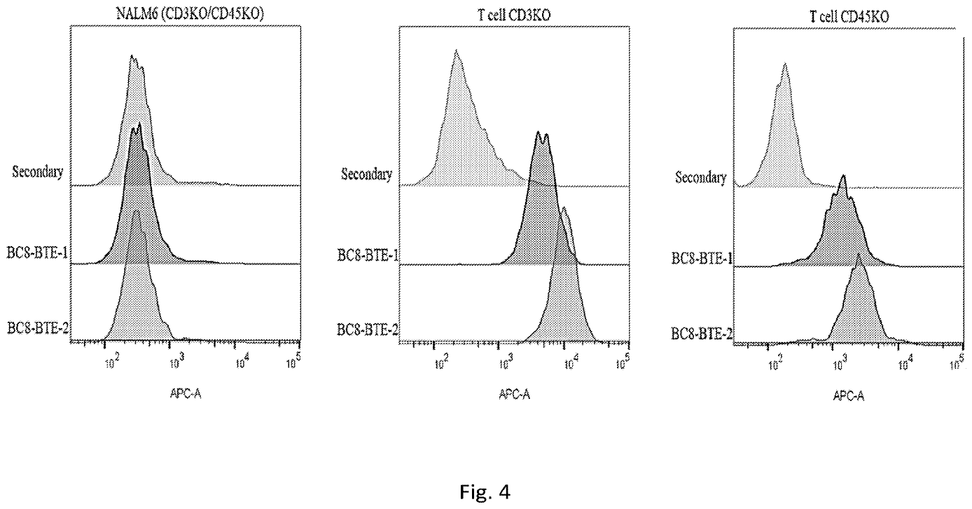

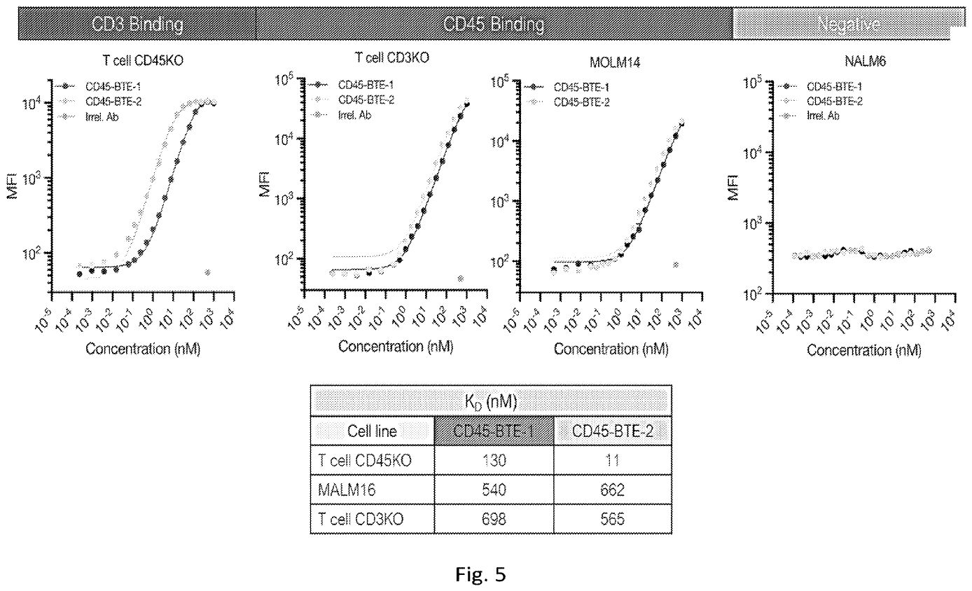

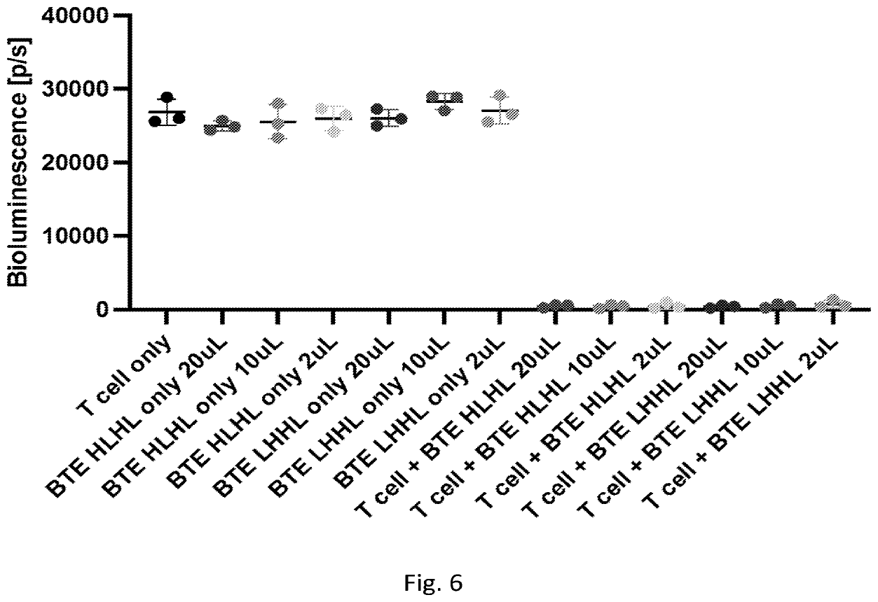

### DETAILED DESCRIPTION

The present invention relates to compositions comprising CD45-specific binding molecules including multi-specific immune cell engaging antibodies (ICE), and CAR molecules, fragments thereof, variants thereof, combinations thereof and nucleic acid molecules encoding the same.

In various embodiments, the multi-specific ICE comprises a binding arm specific for binding to an immune cell specific receptor molecule. In one embodiment, the immune e cell specific receptor molecule is an immune cell surface antigen.

Examples of immune cells that can be targeted by a multi-specific CD45-ICE of the invention include, but are not limited to, T cells, B cells, natural killer (NK) cells, or macrophages.

In some embodiments, the immune cell is a T cell. In one embodiment, the T cell specific receptor molecule is one of CD3, TCR, CD28, CD16, NKG2D, Ox40, 4-1BB, CD2, CD5, CD40, FcgRs, FceRs, FcaRs and CD95. In one embodiment, the T cell specific receptor molecule is CD3.

In some embodiments, the immune cell is a NK cell. In one embodiment, the NK cell receptor molecule is an Fc receptor (FcR) molecule, FasL, sialic acid-bind immunoglobulin-like lectins (Siglecs). NKp30, NKp44. NKp46, NKG2D, DNAX accessory molecule-1 (DNAM-1), 2B4, NTB-A, CD59, NKp80, CD2, and CD94/NKG2C2.

In various embodiments, the antigen binding domain comprises a nucleotide sequence encoding an antibody, a fragment thereof, or a variant thereof specific for binding to an antigen. In one embodiment, the antibody or fragment thereof is a DNA encoded monoclonal antibody (DMAb) or a fragment or variant thereof. In one embodiment, the antibody or fragment thereof is an mRNA encoded monoclonal antibody or a fragment or variant thereof.

In one embodiment, the antigen binding domain of the multi-specific ICE is specific for binding a target antigen, and recruiting an immune cell to the target antigen. In one embodiment, the target antigen is a tumor antigen. In one embodiment, the target antigen is CD45. Therefore, in one embodiment, the invention provides compositions comprising one or more multi-specific ICE and methods for use in treating or preventing cancer or a disease or disorder associated with cancer in a subject.

In one embodiment, the invention relates to CAR T cells expressing CAR molecules comprising an antigen binding domain specific for binding to CD45. In some embodiments, the CD45 binding domain comprises an scFv antibody fragment specific for binding to CD45. In some embodiments, the scFv comprises a sequence as set forth in SEQ ID NO:3. In some embodiment, the CAR molecules of the invention provides for both co-stimulation by CD28 and/or 4-1BB domains, and activation, by a CD3ζ signaling domain.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering an antibody of the present invention to a subject prior to onset of the disease. Suppressing the disease involves administering a antibody of the present invention to a subject after induction of the disease but before its clinical appearance. Repressing the disease involves administering an antibody of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Epitope Editing Combination Therapy

The invention relates, in part to provides methods of use of multi-specific ICEs in combination with methods for epitope editing of healthy cells to reduce cross-reactive binding and targeting of healthy cells. This enables one to unlock and leverage normally expressed proteins for antibody targeting, while diminishing off-target effects to healthy cells like the effector cells. As such, the invention relates, in part, to methods of use of a combination of multi-specific antibody and epitope editing technique for the treatment of possible diseases and disorders.

In some embodiments, the method includes modification of an epitope. The modification to the epitope can be any mutation that reduces clone binding. In some embodiments, the mutation is an insertion, deletion, or point mutation. In some embodiments, the epitope edit is a combination thereof. In some embodiments, the insertion is one or more amino acid at one or more positions. In some embodiments, the point mutation is a replacement of one or more amino acid for any other amino acid. In some embodiments, the point mutation is an alanine substitution.

In some embodiments, the method includes epitope editing of healthy cells from a subject or patient. In some embodiments, the healthy cells to be epitope-edited for the patient are blood cells and hematopoietic stem cells. The blood cells can be white blood cells or red blood cells. The white blood cells can be immune effector cells. In some embodiments, the white blood cells are T cells, B cells, NK cells, NK-T cells, neutrophils, macrophages, monocytes, dendritic cells. In some embodiments, T cells are edited alongside the hematopoietic stem cells.

In some embodiments, the editing of healthy cells can take place ex vivo or directly in the patient. In some embodiments, when the editing of healthy cells takes place ex vivo, the immune cells are transplanted into the patient. In some embodiments, the cells are edited ex vivo before transplant. In some embodiments, the ex vivo edited donor immune effector cells and hematopoietic stem cells are from an autologous or allogeneic source for transplant.

The method of epitope editing used for healthy cells to ablate cross-reactive antibody targeting that can be employed in the methods of the invention can be any genome therapy technique. Exemplary methods of epitope editing include, but are not limited to, restriction enzyme based editing, zinc finger based editing, TALENS, CRISPR/Cas9, base editing using a CRISPR based system, prime editing, PASTE, and meganuclease based editing.

Restriction enzyme based editing generally involves the use of restriction enzymes that recognize specific patterns of nucleotide sequences and cut at that site, presenting an opportunity to insert new DNA material at that location.

Zinc finger based editing is generally composed of two parts: an engineered nuclease (Fokl) fused to zinc finger DNA-binding domains. The zinc-finger DNA-binding domain recognizes a 3-base pair site on DNA and can be combined to recognize longer sequences.

TALENS based editing generally comprise a non-specific DNA-cleaving nuclease fused to a DNA-binding domain that can be easily engineered so that TALENs can target essentially any sequence.

CRISPR/Cas9 based editing is generally a two-component system consisting of a guide RNA and a Cas9 nuclease. The Cas9 nuclease cuts the DNA within the ~20 nucleotide region defined by customized guide RNAs.

Base editing generally uses components from CRISPR systems together with other enzymes to directly install point mutations into cellular DNA or RNA without making double-stranded DNA breaks (DSBs). In some embodiments, DNA base editors comprise a catalytically disabled nuclease fused to a nucleobase deaminase enzyme and, in some cases, a DNA glycosylase inhibitor.

Prime editing generally uses a single engineered construct known as a prime editing guide RNA (pegRNA), which is made up of the primer binding site (PBS) sequence and a sequence containing the desired edit. After it finds the target, the Cas9 nickase creates a cut in one strand of the DNA and the reverse transcriptase uses the pegRNA as a template for reverse transcription, attaching the corresponding nucleotides to the nicked DNA end.

PASTE editing generally incorporates prime editing to copy AttB sites into the genome near a target sequence via reverse transcription. Once the AttB site has been incorporated near the target sequence via prime editing, it acts as a beacon for the integrase, which will be recruited to the site to perform the insertion of the attached desired DNA sequence.

Meganuclease editing generally takes advantage of the endonuclease activity, which allows surrounding introns and inteins to act as invasive DNA elements, while the splicing activity allows the endonuclease gene to invade a coding sequence without disrupting its product. The high specificity of these enzymes is based on their ability to cleave dsDNA at specific recognition sites comprising 14-40 bp.

In some embodiments, the methods of the invention generally include methods of epitope editing of CD45, CD3, CD2, CD5, CD7, CD19, CD20, CD22, CD33, CCR5, CCR8, or BCMA. In some embodiments, the methods of the invention include a combination of epitope editing of CD45, CD3, CD2, CD5, CD7, CD19, CD20, CD22, CD33, CCR5, CCR8, or BCMA and further administering a multi-specific antibody specific for binding to CD45, CD3, CD2, CD5, CD7, CD19, CD20, CD22, CD33, CCR5, CCR8, or BCMA.

In some embodiments, the multispecific antibody can be bispecific or trispecific. In bispecific embodiments, the antibody can be bivalent with one binding domain per antigen. In trispecific embodiments, the antibody can be trivalent with one binding domain per antigen.

In some embodiments, the invention provides a combination therapy comprising epitope editing of CD45, CD3, CD2, CD5, CD7, CD19, CD20, CD22, CD33, CCR5, CCR8, or BCMA in combination with a multi-specific antibody targeting CD45, CD3, CD2, CD5, CD7, CD19, CD20, CD22, CD33, CCR5, CCR8, or BCMA or a combination thereof. The modification to the epitope can be any mutation that reduces clone binding. In some embodiments, the mutation is an insertion, deletion, or point mutation. In some embodiments, the epitope edit is a combination thereof. In some embodiments, the insertion is one or more amino acid at one or more positions. In some embodiments, the point mutation is a replacement of one or more amino acid for any other amino acid. In some embodiments, the point mutation is an alanine substitution.

In some embodiments, the invention provides compositions comprising a modification to CD45 to edit an epitope that is recognized by an antibody or CD45 binding molecule. In some embodiments, the edit of the epitope is at positions D229-Y232, V258-E259, T266, N267, I283, H285, or N286 of CD45. In one embodiment, the epitope edit is Y232C of CD45.

In some embodiments, the methods of the invention are used to reduce an antibody clone's binding and detection of healthy cells. Therefore, in some embodiments, the method is used to introduce a modification into a healthy cell that allows the cell to avoid detection by an antibody when administered in combination with an antibody that targets the unmodified version of the epitope in disease-associated cells. In some embodiments, the method includes the use of a multi-specific antibody or multi-specific ICE to target T cells to disease associated cells.

In some embodiments, the epitope editing results in reduction of epitope binding by a co-administered multi-specific antibody by at least 70%, 75%, 80%, 85%, 90%, 95%, 98%, 99% or greater than 99% as compared to the level of binding by the antibody to the un-modified epitope.

In some embodiments, the epitope-directed genome editing method of the invention is combined with one or more immune cell genome edits. The additional genome edits can enhance efficacy, delay exhaustion, improve safety, prevent fratricide, or any combination thereof. Exemplary additional gene edits that can be combined with the methods of the invention include, but are not limited to, editing of FAS, PD-1, PDL-1, TGFBR, or CD7.

The methods of the invention can be used for the treatment of any disease or disorder that can be treated using antibody-based immunotherapeutics. In some embodiments, the method comprises administering a multi-specific antibody targeting a disease associated protein or antigen, in combination with engineered cells, wherein the engineered cells comprise a modification in one or more epitope targeted by the multi-specific antibody to reduce binding of the multi-specific antibody to the engineered cell.

In some embodiments, the multi-specific antibody targets an epitope of a protein or antigen associated with cancer, an autoimmune disease or disorder, or an infectious disease or disorder. Therefore, in some embodiments, the invention provides methods of treating or preventing cancer, an autoimmune disease or disorder, or an infectious disease or disorder by administering a multi-specific antibody in combination with one or more engineered cell comprising at least one modification of one or more epitope targeted by the multi-specific antibody. In some embodiments, the invention provides methods of treating or preventing cancer, an autoimmune disease or disorder, or an infectious disease or disorder by administering a multi-specific antibody in combination with one or more compositions for epitope editing of one or more epitope targeted by the multi-specific antibody.

### Compositions

In one embodiment, the present invention relates to compositions comprising multi-specific ICE (e.g., a bispecific immune cell engager (or BICE) or bispecific T cell engager (or BiTE)), a fragment thereof, a variant thereof, or a combination thereof. The compositions, when administered to a subject in need thereof, can result in the generation of a synthetic bispecific immune cell engager in the subject. In some embodiments, the multi-specific ICE of the invention is encoded by a nucleic acid molecule (e.g., DNA or mRNA). Therefore, in some embodiments, the invention relates to nucleic acid molecules encoding a multi-specific ICE.

In one embodiment, the multi-specific ICE comprises at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB. CD2, CD5, CD40, FcgRs, FceRs, FcaRs and CD95.

In some embodiments, the invention provides chimeric antigen receptor (CAR) molecules comprising an antigen binding domain and an immune cell activation domain. In some embodiments, the immune cell activation domain comprises at least one intracellular domain from a co-stimulatory molecules, for example, CD28, 4-1BB, ICOS, OX40, and the like.

In various embodiments, the antigen binding domain comprises an antibody, a fragment thereof (e.g., an scFv fragment, an scFv-Fc fragment, a tandem scFv-Fc or a tandem scFc), or a variant thereof specific for binding to an antigen. In one embodiment, the antigen is a tumor antigen. In one embodiment, the antigen is CD45, CD3, CD2, CD5, CD7, CD19, CD20, CD22, CD33, CCR5, CCR8, or BCMA.

In certain embodiments, the composition can treat, prevent, and or/protect against a disease or disorder associated with CD45, CD3, CD2, CD5, CD7, CD19, CD20, CD22, CD33, CCR5, CCR8, or BCMA expression.

In one embodiment, the multi-specific binding molecule is specific for binding to CD45. Exemplary CD45 binding molecules include, but are not limited to, binding molecules that comprises a variable heavy chain of SEQ ID NO:1, or a fragment or variant thereof and a variable light chain of SEQ ID NO:2, or a fragment or variant thereof. In some embodiments, the variable heavy and variable light chain are oriented in the heavy-light (HL) orientation. In some embodiments, the variable heavy and variable light chain are oriented in the light-heavy (LH) orientation. In some embodiments, the CD45 binding molecule comprises the amino acid sequence of SEQ ID NO:5, SEQ ID NO:7, SEQ ID NO:11, SEQ ID NO:12, SEQ ID NO:13, SEQ ID NO:14, SEQ ID NO: 19, SEQ ID NO:20, SEQ ID NO:21, SEQ ID NO:22, SEQ ID NO:23, SEQ ID NO:24, SEQ ID NO:25, SEQ ID NO:26. SEQ ID NO:27 or SEQ ID NO:28 or a fragment or variant thereof. In one embodiment, the fragment or variant of SEQ ID NO:5, SEQ ID NO:7, SEQ ID NO:11, SEQ ID NO:12, SEQ ID NO:13, SEQ ID NO:14, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 conserves the CDR sequences of the variable heavy and variable light chains.

In various embodiments, the antigen binding domain comprises a binding arm specific for CD45. In some embodiments, the CD45 binding arm comprises an amino acid sequence as set forth in SEQ ID NO:5 or SEQ ID NO:7.

In certain embodiments, the composition can treat, prevent, and or/protect against a disease or disorder associated with CD45 expression. In certain embodiments, the composition can treat, prevent, and or/protect against cancer associated with CD45 expression.

The synthetic antibody (e.g., multi-specific ICE) or the CAR molecule of the invention, can treat, prevent, and/or protect against disease in the subject administered the composition. The synthetic antibody (e.g., multi-specific ICE) or the CAR molecule of the invention can promote survival of the disease in the subject administered the composition. The synthetic antibody (e.g., multi-specific ICE) or the CAR molecule can provide at least about 50%, 55%, 60%, 65%, 70%, 75%, 80%, 85%, 90%, 95%, or 100% survival of the disease in the subject administered the composition. In other embodiments, the synthetic antibody (e.g., multi-specific ICE) or the CAR molecule can provide at least about 65%, 66%, 67%, 68%, 69%, 70%, 71%, 72%, 73%, 74%, 75%, 76%, 77%, 78%, 79%, or 80% survival of the disease in the subject administered the composition.

The composition can result in the generation of the synthetic multi-specific binding molecule (e.g., multi-specific ICE) or the CAR molecule in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic multi-specific binding molecule (e.g., multi-specific ICE) in the subject within at least about 1 day, 2 days, 3 days, 4 days, 5 days, 6 days, 7 days, 8 days, 9 days, or 10 days of administration of the composition to the subject. The composition can result in generation of the synthetic multi-specific binding molecule (e.g., multi-specific ICE) or the CAR molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic multi-specific binding molecule (e.g., multi-specific ICE) or the CAR molecule in the subject more quickly than the generation of an endogenous antibody in a subject who is administered an antigen to induce a humoral immune response. The composition can result in the generation of the synthetic multi-specific binding molecule (e.g., multi-specific ICE) or the CAR molecule at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### Antibody

In some embodiments, the invention relates to an antibody, a fragment thereof, a variant thereof, or a combination thereof. The antibody can bind or react with an antigen, which is described in more detail below. In some embodiments, the antibody is a multi-specific antibody which can bind or react with two or more antigen, which is described in more detail below. In some embodiments the fragment is an scFv fragment, an scFv-Fc fragment, a tandem scFv-Fc or a tandem scFv. In some embodiments, the antibody is a bispecific immune cell engager (BiICE), a fragment thereof, or a variant thereof. In some embodiments, the antibody is a trispecific immune cell engager, a fragment thereof, or a variant thereof. In some embodiments, the antibody is a multispecific immune cell engager, a fragment thereof, or a variant thereof comprising at least one domain specific for binding to an immune cell and one or more domains specific for binding to CD45. In some embodiments, the antibody is a bispecific T cell engagers (BiTE), a fragment thereof, or a variant thereof. In some embodiments, the antibody is a trispecific T cell engager, a fragment thereof, or a variant thereof. In some embodiments, the antibody is a multispecific immune cell engager, a fragment thereof, or a variant thereof comprising at least one domain specific for binding to an CD3 and one or more domains specific for binding to CD45. In some embodiments, the antibody is a multispecific immune cell engager, a fragment thereof, or a variant thereof comprising at least one domain specific for binding to an CD3 and two domains specific for binding to CD45.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD. IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

The antibody may have a half-life within the subject. In some embodiments, the antibody may be modified to extend or shorten its half-life within the subject. Such modifications are described below in more detail.

### Heavy Chain Polypeptide

The binding molecule of the invention can include a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### Light Chain Polypeptide

The binding molecule of the invention can include a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### Linker Sequence

The binding molecule of the invention of the invention can include one or more linker sequences. The linker sequence can spatially separate or link the one or more components described herein. In other embodiments, the linker sequence can comprise an amino acid sequence that spatially separates or links two or more polypeptides. In one embodiment, the linker sequence is a G4S linker sequence.

### Leader Sequence

The binding molecule of the invention of the invention can include one or more leader sequences. In one embodiment, the leader sequence is a signal peptide. The signal peptide can be an immunoglobulin (Ig) signal peptide, for example, but not limited to, an IgG signal peptide and a IgE signal peptide.

### ScFv Antibody

In one embodiment, the binding molecule of the invention comprises a scFv antibody fragment. In one embodiment, scFv relates to a Fab fragment without the CH1 and CL regions. Thus, in one embodiment, the scFv relates to a Fab fragment comprising the VH and VL. In one embodiment, the scFv comprises a linker between VH and VL. In one embodiment, the scFv relates to a Fab fragment comprising the VL and VH. In one embodiment, the scFv comprises a linker between VL and VH. In one embodiment, the ScFv fragment of the invention has modified expression, stability, half-life, antigen binding, heavy chain-light chain pairing, tissue penetration or a combination thereof as compared to a parental antibody.

In one embodiment, the scFv of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental antibody.

### ScFc Antibody

In some embodiments, the multi-specific ICE of the invention comprises at least one Fc region. In some embodiments, the multi-specific ICE of the invention comprise a single chain Fc region (“scFc”) in which the component Fc moieties are genetically-fused in a single polypeptide chain such that they form a functional, dimeric Fc region. In certain embodiments, the component Fc moieties of an scFc are genetically fused in tandem via a polypeptide linker (e.g., an Fc connecting peptide) interposed between the Fc moieties. Thus, the scFc polypeptides of the invention comprise scFc region(s) formed by a single contiguous amino acid sequence. In certain aspects, the Fc regions (i.e., scFc region) of a scFc polypeptide may be operably linked to the binding site of a binding polypeptide (e.g., to an antigen binding fragment (e.g., a Fab) or an scFv molecule) to form a scFc binding polypeptide, thereby imparting an effector function to the binding polypeptide or altering an existing effector function. scFc multi-specific ICEs of the invention may be monomeric or multimeric (e.g., dimeric). In one embodiment, the binding molecule of the invention comprises a single chain Fc (scFc) antibody fragment. In some embodiments, the single chain Fc comprises CH2 and CH3 domains.

In one embodiment, the binding molecule of the invention comprises a single chain Fv-Fc (scFv-Fc) antibody fragment or a tandem Fc antibody fragment. In one embodiment, single chain Fv-Fc relates to a single chain Fab fragment in which an scFv fragment is fused to the hinge, CH2, and CH3 domains. Thus, in one embodiment, the scFv-Fc relates to a Fab fragment comprising the VH and VL and further comprising the hinge, CH2, and CH3 domains. In one embodiment, the scFv-Fc comprises a linker between VH and VL and one or more additional linker between the scFv and the Fc region comprising the hinge, CH2, and CH3 domains. In one embodiment, the scFv-Fc antibody fragment of the invention has modified expression, stability, half-life, antigen binding, heavy chain-light chain pairing, tissue penetration or a combination thereof as compared to a parental antibody.

In one embodiment, the multi-specific antibody is a tandem scFc antibody comprising tandem Fc domains. For example in one embodiment, the tandem scFc antibody comprises a CH2-CH3-CH2-CH3 monomer linked in a single chain. Exemplary linkers that can be used in the tandem scFc, but are not limited to, helical linkers and flexible linkers.

In one embodiment, the antibody or binding molecule of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental antibody.

In one embodiment, the antibody or binding molecule of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental antibody.

In one embodiment, the antibody or binding molecule of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental antibody.

In one embodiment, the antibody or binding molecule of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental antibody.

In one embodiment, the antibody or binding molecule of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental antibody.

In one embodiment, the antibody or binding molecule of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental antibody.

### Multi-Specific Antibody

The multi-specific ICE of the invention can be a bispecific antibody or binding molecule, a trispecific antibody or binding molecule, a fragment thereof, a variant thereof, or a combination thereof. The bispecific antibody or binding molecule can bind or react with two antigens, for example, two of the antigens described below in more detail. The trispecific antibody or binding molecule can bind or react with three antigens, for example, three of the antigens described below in more detail. The multi-specific antibody or binding molecule can be comprised of fragments of two or more of the antibodies described herein, thereby allowing the multi-specific antibody to bind or react with two or more desired target molecules, which may include the antigen, which is described below in more detail, a ligand, including a ligand for a receptor, a receptor, including a ligand-binding site on the receptor, a ligand-receptor complex, and a marker.

The invention provides novel multi-specific binding molecules comprising a first antigen-binding site that specifically binds to a first target and at least one additional antigen-binding site that specifically binds to at least one additional target, with particularly advantageous properties such as producibility, stability, binding affinity, biological activity, specific targeting of certain cell populations (e.g., T cells or NK cells), targeting efficiency and reduced toxicity. In some instances, there are multi-specific antibodies, wherein multi-specific antibody binds to a first target with high affinity and to at least one additional target with low affinity. In other instances, there are multi-specific antibodies, wherein the multi-specific antibody binds to the first target and to one or more additional target with high affinity.

In one embodiment, the multi-specific binding molecule is a tandem scFv antibody or a tandem scFV-Fc antibody comprising a) a first scFv or scFv-Fc specific for binding to a first antigen, and b) one or more additional scFv or scFv-Fc specific for binding to one or more additional antigen. In some embodiments the tandem scFv or tandem scFv-Fc comprises a linker sequence between each of the scFv or scFv-Fc. Exemplary linkers that can be used in the tandem scFv or scFv-Fc include, but are not limited to, helical linkers and flexible linkers.

The multi-specific binding molecule according to the invention may have two or more epitope binding domains. In some embodiments, one of the epitope binding domains is capable of binding to a tumor antigen. In some embodiments, one of the binding sites of the multi-specific binding molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing .alpha.- and .beta.-chains, in some embodiments, it encompasses .gamma.-chains and .delta.-chains (supra). Accordingly, in some embodiments, the TCR is TCR (alpha/beta) and in some embodiments, it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments, a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the multi-specific binding molecule binds a tumor antigen and the second binding site binds a T cell specific receptor molecule and/or a natural killer (NK) cell specific receptor molecule.

In some embodiments, the first binding site of the multi-specific binding molecule binds CD45, CD3, CD2, CD5, CD7, CD19, CD20, CD22, CD33, CCR5, CCR8, or BCMA, and the second binding site binds a T cell specific receptor molecule and/or a natural killer (NK) cell specific receptor molecule. In some embodiments, the first binding site of the antibody molecule binds CD45, CD3, CD2, CD5, CD7, CD19, CD20, CD22, CD33, CCR5, CCR8, or BCMA and the second binding site binds one of CD3, TCR, CD28, CD16, NKG2D, Ox40, 4-1BB, CD2, CD5, CD40, FcgRs, FceRs, FcaRs and CD95. In some embodiments, the first binding site of the antibody molecule binds CD45 and the second binding site binds CD3.

In some embodiments, the first binding site of the antibody molecule binds a T cell specific receptor molecule and/or a natural killer (NK) cell specific receptor molecule and the second binding site binds a tumor antigen. In some embodiments, the first binding site of the antibody binds a T cell specific receptor molecule and/or a natural killer (NK) cell specific receptor molecule and the second binding site binds CD45, CD3, CD2, CD5, CD7, CD19, CD20, CD22, CD33, CCR5, CCR8, or BCMA. In some embodiments, the first binding site of the antibody binds one of CD3, TCR, CD28, CD16, NKG2D, Ox40, 4-1BB, CD2, CD5, CD40, FcgRs, FceRs, FcaRs and CD95, and the second binding site binds CD45, CD3, CD2, CD5, CD7, CD19, CD20, CD22, CD33, CCR5, CCR8, or BCMA. In some embodiments, the first binding site of the multi-specific binding molecule binds CD3, and the second binding site binds CD45.

In one embodiment the multi-specific antibody of the invention comprises a BiICE, comprising two tandem scFv-Fc antibody fragments as described herein, allowing the BiICE to bind or react with the desired target molecules. In one embodiment the bispecific antibody of the invention comprises a BiICE, comprising two tandem scFv-Fc antibody fragments as described herein, allowing the BiICE to bind or react with the desired target molecules. In one embodiment the bispecific antibody of the invention comprises a BiICE, comprising three tandem scFv-Fc antibody fragments as described herein, allowing the BiICE to bind or react with the desired target molecules.

In one embodiment the BiICE, comprises a first scFv-Fc specific for binding to a target disease-specific antigen linked to a second scFv-Fc specific for binding to an immune cell specific receptor molecule. In one embodiment the BiICE, comprises a first scFv-Fc specific for binding to a target disease-specific antigen linked to a second scFv-Fc specific for binding to an immune cell specific receptor molecule, linked to a third scFv-Fc specific for binding to the target disease-specific antigen. The linkage may place the domains in any order, for example, in one embodiment, a nucleotide sequence encoding a scFv-Fc specific for binding to a target disease-specific antigen is oriented C-Terminal to a scFv-Fc specific for binding to a T cell specific receptor molecule. In one embodiment, a nucleotide sequence encoding a scFv-Fc specific for binding to a target disease-specific antigen is oriented C-Terminal to a scFv-Fc specific for binding to a T cell specific receptor molecule, which is oriented C-Terminal to a scFv-Fc specific for binding to the target disease-specific antigen. In another embodiment, a nucleotide sequence encoding a scFv-Fc specific for binding to a target disease-specific antigen is oriented N-Terminal to a nucleotide sequence encoding a scFv-Fc specific for binding to a T cell specific receptor molecule.

### Multi-Functional Antibody

The multi-specific T cell engager can be a multi-functional antibody, a fragment thereof, a variant thereof, or a combination thereof. The multi-functional antibody can bind or react with two or more (e.g., 2, 3, 4, 5 or more than 5) antigens as described below. The multi-functional antibody can also be modified to impart an additional functionality to the antibody beyond recognition of and binding to the antigens. Such a modification can include, but is not limited to, coupling to factor H or a fragment thereof. Factor H is a soluble regulator of complement activation and thus, may contribute to an immune response via complement-mediated lysis (CML).

### Bispecific Immune Cell Engager

As described above, the binding molecule of the invention can be a bispecific immune cell engager (BiICE), a fragment thereof, a variant thereof, or a combination thereof. The antigen targeting domain of the BiICE can bind or react with the antigen, which is described in more detail below.

The antigen targeting domain of the BiICE may comprise an antibody, a fragment thereof, a variant thereof, or a combination thereof. The antigen targeting domain of the BiICE may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding domain, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antigen targeting domain of the BiICE can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antigen targeting domain of the BiICE can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antigen targeting domain of the BiICE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

In some embodiments, the invention includes nucleic acid molecules (e.g., DNA or mRNA) encoding the BiICE.

In one embodiment, at least one of the antigen binding domain and the immune cell engaging domain of the BiICE of the invention is an scFv antibody fragment as described in detail above.

### Multi-Valent Bispecific Immune Cell Engager

The bispecific immune cell engager (BiICE) of the invention may be a multi-valent BiICE, comprising two or more antigen targeting domains specific for the same target antigen. Each of the two or more antigen targeting domain of the multi-valent BiICE may comprise an antibody, a fragment thereof, a variant thereof, or a combination thereof. The antigen targeting domain of the BiICE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule. In one embodiment, at least one of the antigen binding domain and the immune cell engaging domain of the BiICE of the invention is an scFv or tandem Fc domain fragment as described in detail above.

In some embodiments, the multi-valent BiICE comprises a combination of a first and a second binding domain for targeting CD45. In some embodiments, the multi-valent BiICE comprises a combination of a first and a second binding domain for targeting CD45 and a binding domain for targeting an immune cell. In some embodiments, the multi-valent BiICE comprises a combination of a first binding domain for targeting CD45, a second binding domain for targeting an immune cell, and a binding domain for targeting CD45, and

In some embodiments, the invention includes one or more nucleic acid molecules (e.g., DNA or mRNA) encoding the multi-valent BiICE.

### Extension of Antibody Half-Life

The synthetic antibody (e.g., multi-specific ICE) may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions. The modification may be a linked Fc domain.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Antigen

In one embodiment, the synthetic antibody (e.g., multi-specific ICE) or CAR molecule is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be a tumor antigen. The antigen can be associated with increased risk of cancer development or progression. In one embodiment, the antigen can be CD45, CD3, CD2, CD5, CD7, CD19, CD20, CD22, CD33, CCR5, CCR8, or BCMA.

In one embodiment, a synthetic bispecific immune cell engager of the invention targets two or more antigens. In one embodiment, at least one antigen of a bispecific antibody is a tumor antigen. In one embodiment, at least one antigen of a bispecific antibody is a T-cell activating antigen.

### Tumor Antigen

The antigen binding domain of the synthetic antibody (e.g., multi-specific ICE) or CAR molecule of the invention can interact with a tumor antigen. In the context of the present invention, “tumor antigen” or “hyperproliferative disorder antigen” or “antigen associated with a hyperproliferative disorder,” refers to antigens that are common to specific hyperproliferative disorders such as cancer.

The type of tumor antigen referred to in the invention may be a tumor-specific antigen (TSA) or a tumor-associated antigen (TAA). A TSA is unique to tumor cells and does not occur on other cells in the body. A TAA antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are proteins that are produced by tumor cells. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase. RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, CD123, B-cell maturation antigen (BCMA), Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2, Her3, IGFR, CD133, IL3R, fibroblast activating protein (FAP), CDCP1, Derlinl. Tenascin, frizzled 1-10, the vascular antigens VEGFR2 (KDR/FLK1), VEGFR3 (FLT4, CD309), PDGFR-α (CD140a), PDGFR-.beta. (CD140b) Endoglin, CLEC14, Tem1-8, and Tie2. Further examples may include A33, CAMPATH-1 (CDw52), Carcinoembryonic antigen (CEA). Carboanhydrase IX (MN/CA IX), CD21, CD25, CD30, CD34, CD37, CD44v6, CD45, CD133, de2-7 EGFR, EGFRvIII, EpCAM, Ep-CAM, Folate-binding protein, G250, Fms-like tyrosine kinase 3 (FLT-3, CD135), follicle stimulating hormone receptor (FSHR), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA). Prostate stem cell antigen (PSCA), Prostate specific antigen (PSA), and TAG-72. Examples of antigens expressed on the extracellular matrix of tumors are tenascin and the fibroblast activating protein (FAP).

In one embodiment, the tumor antigen is a hormone or fragment thereof which can be used to target a specific receptor. Examples include, but are not limited to, FSH hormone, LH hormone, TSH hormone or fragments thereof.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations: such as BCR-ABL. E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4. MAGE-5, MAGE-6. RAGE, NY-ESO, p185erbB2, p180erbB-3, c-met, nm-23H1, PSA, TAG-72, CA 19-9. CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16. TA-90\Mac-2 binding protein\cyclophilin C-associated protein, TAAL6, TAG72, TLP, and TPS.

Aspects of the present invention include compositions for enhancing an immune response against an antigen in a subject in need thereof, comprising a synthetic antibody (e.g., multi-specific ICE) or CAR molecule capable of generating an immune response in the subject, or a biologically functional fragment or variant thereof. In some embodiments, the antigen is CD45, CD3, CD2, CD5, CD7, CD19, CD20, CD22, CD33, CCR5, CCR8, or BCMA. In some embodiments, the synthetic antibody of this invention is a BiICE comprising at least one scFv-Fc targeting CD45.

### T Cell Specific Receptor

In one embodiment, the multi-specific ICE of the invention comprises a scFv or scFv-Fc specific for binding to a T cell specific receptor. T cell specific receptors include, but are not limited to, CD3. TCR. CD28, CD16, NKG2D, Ox40, 4-1BB, CD2, CD5, CD40, FcgRs, FceRs, FcaRs and CD95.

### CAR Molecules

In one embodiment, the invention provides a chimeric antigen receptor (CAR) comprising an antigen binding domain and a T cell activation domain. In one embodiment, the antigen binding domain is a targeting domain, wherein the targeting domain directs the cell expressing the CAR to a cell or particle expressing the antigen. In one embodiment, the antigen is CD45, CD3, CD2, CD5, CD7, CD19, CD20, CD22, CD33, CCR5, CCR8, or BCMA.

In various embodiments, the CAR can be a “first generation,” “second generation,” “third generation,” “fourth generation” or “fifth generation” CAR (see, for example. Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3 chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is a CD45 scFv antibody fragment, or a variant thereof, specific for binding to CD45. In some embodiments the CD45 binding domain comprises a sequence as set forth in SEQ ID NO:5 or SEQ ID NO:7.

In various embodiments, the CAR molecules of the invention provides for both co-stimulation by CD28 and/or 4-1BB domains, and activation, by a CD3ζ signaling domain.

### Substrates

In one embodiment, the present invention provides a scaffold, substrate, or device comprising a bispecific immune cell engager, fragment thereof, or nucleic acid molecule encoding the same. For example, in some embodiments, the present invention provides a tissue engineering scaffold, including but not limited to, a hydrogel, electrospun scaffold, polymeric matrix, or the like, comprising the modulator. In certain embodiments, a bispecific immune cell engager, fragment thereof, or nucleic acid molecule encoding the same, may be coated along the surface of the scaffold, substrate, or device. In certain embodiments, the bispecific immune cell engager, fragment thereof, or nucleic acid molecule encoding the same is encapsulated within the scaffold, substrate, or device.

### Recombinant Nucleic Acid Sequence

As described above, the composition can comprise a recombinant nucleic acid sequence. The recombinant nucleic acid sequence can encode the synthetic antibody (e.g., multi-specific ICE) or the CAR molecule, a fragment thereof, a variant thereof, or a combination thereof.

The recombinant nucleic acid sequence can be a heterologous nucleic acid sequence. The recombinant nucleic acid sequence can include at least one heterologous nucleic acid sequence or one or more heterologous nucleic acid sequences.

In one embodiment, a nucleotide sequence encoding a CD45-BiICE comprises a nucleotide sequence of SEQ ID NO:11, SEQ ID NO:12, SEQ ID NO:13, SEQ ID NO:14, SEQ ID NO:19, SEQ ID NO:20, SEQ ID NO:21, SEQ ID NO:22, SEQ ID NO:23. SEQ ID NO:24, SEQ ID NO:25, SEQ ID NO:26, SEQ ID NO:27 or SEQ ID NO:28 or a fragment or variant thereof. In one embodiment, a nucleotide sequence encoding a CD45-BiICE comprises an RNA sequence corresponding to a DNA sequence as set forth in SEQ ID NO:15, SEQ ID NO:16, SEQ ID NO:17 or SEQ ID NO:18 or a fragment or variant thereof.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence. Accordingly, in one embodiment, the invention provides an RNA molecule encoding one or more of the synthetic antibodies of the invention. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively. UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement I as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody (e.g., multi-specific ICE) or the CAR molecule. In particular, the heavy chain polypeptide and the light chain polypeptide can interact with one another such that assembly results in the synthetic antibody (e.g., multi-specific ICE) or the CAR molecule being capable of binding the antigen. In other embodiments, the heavy chain polypeptide and the light chain polypeptide can interact with one another such that assembly results synthetic antibody (e.g., multi-specific ICE) or the CAR molecule being more immunogenic as compared to an antibody not assembled as described herein. In still other embodiments, the heavy chain polypeptide and the light chain polypeptide can interact with one another such that assembly results in the synthetic antibody (e.g., multi-specific ICE) or the CAR molecule being capable of eliciting or inducing an immune response against the antigen.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Methods of Delivery of the Composition

The present invention also relates to a method of delivering the composition to the subject in need thereof. The method of delivery can include, administering the composition to the subject. In some embodiments, the present invention relates to administration of a bispecific antibody of the invention, scFv. CAR molecule, CAR T cell, or a nucleic acid molecule encoding a bispecific antibody, scFv, or CAR molecule of the invention. In some embodiments, the nucleic acid molecule is a DNA molecule. In some embodiments, the nucleic acid molecule is an RNA molecule. In some embodiments, the nucleic acid molecule is an mRNA molecule.

Administration can include, but is not limited to, intravenous delivery of an antibody, scFv, CAR molecule, or CAR T cell, DNA injection, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Method of Treatment

Also provided herein is a method of treating, protecting against, and/or preventing disease in a subject in need thereof by administration of a bispecific antibody of the invention, scFv, CAR molecule, CAR T cell, or a nucleic acid molecule encoding a bispecific antibody, scFv, or CAR molecule of the invention to the subject. The method can include administering a composition comprising a bispecific antibody of the invention, scFv. CAR molecule, CAR T cell, or a nucleic acid molecule encoding a bispecific antibody, scFv, or CAR molecule of the invention to the subject. Administration of the composition to the subject can be done using the method of delivery described above.

In certain embodiments, the invention provides a method of treating protecting against, and/or preventing cancer. In one embodiment, the method treats, protects against, and/or prevents tumor growth. In one embodiment, the method treats, protects against, and/or prevents cancer progression. In one embodiment, the method treats, protects against, and/or prevents cancer metastasis.

In one embodiment, the invention provides methods for preventing growth of benign tumors, such as, but not limited to, uterine fibroids. The methods comprise administering an effective amount of one or more of the compositions of the invention to a subject diagnosed with a benign tumor.

Upon administration of the synthetic antibody, BiTe, scFv, CAR molecule or CART cell to the subject, the synthetic antibody, BiTe, scFv. CAR molecule or CAR T cell can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 g to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### Cancer Therapy

The invention provides methods of treating or preventing cancer, or of treating and preventing growth or metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a composition of the invention. The invention further provides a method of inhibiting metastasis in an individual in need thereof, the method comprising administering to the individual an effective metastasis-inhibiting amount of any one of the compositions described herein.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, the compositions of the invention can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the compositions of the invention to a subject diagnosed with a malignant tumor or cancerous cells or to a subject having a tumor or cancerous cells.

The following are non-limiting examples of cancers that can be treated by the methods and compositions of the invention include, but are not limited to, blood cancers. Exemplary blood cancers include, but are not limited to leukemia, myeloma, and lymphoma. In one embodiments, the cancer is acute myeloid leukemia (AML).

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase 11 inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU. BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin: altretamine; ambomycin; ametantrone acetate; aminoglutethimide; amsacrine; anastrozole; anthramycin; asparaginase; asperlin; azacitidine: azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin: cedefingol; chlorambucil; cirolemycin; cisplatin: cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin: enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine: fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b: iproplatin; irinotecan hydrochloride; lanreotide acetate: letrozole; leuprolide acetate: liarozole hydrochloride; lometrexol sodium; lomustine: losoxantrone hydrochloride: masoprocol; maytansine: mechlorethamine hydrochloride; megestrol acetate: melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper: mitotane; mitoxantrone hydrochloride; mycophenolic acid; nocodazole; nogalamycin; ormaplatin; oxisuran; paclitaxel; pegaspargase: peliomycin; pentamustine; peplomycin sulfate; perfosfamide; pipobroman; piposulfan; piroxantrone hydrochloride; plicamycin; plomestane; porfimer sodium: porfiromycin; prednimustine; procarbazine hydrochloride; puromycin: puromycin hydrochloride; pyrazofurin; riboprine; rogletimide; safingol; safingol hydrochloride: semustine: simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine: thiotepa; tiazofurin; tirapazamine; toremifene citrate: trestolone acetate; triciribine phosphate: trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin: zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide: angiogenesis inhibitors; antagonist D; antagonist G: antarelix; anti-dorsalizing morphogenetic protein-1: antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane: buthionine sulfoximine; calcipotriol; calphostin C; camptothecin derivatives; canarypox IL-2; capecitabine; carboxamide-amino-triazole; carboxyamidotriazole: CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

Agents that Improve T Cell Response

In some aspects, the synthetic antibody, scFv, multi-specific ICE, CAR construct or CAR T cell is administered in combination with at least one agent that improves T cell response. In some embodiments, at least one agent that improves T cell response is an immune checkpoint inhibitor. In some aspects, the immune check point inhibitor used in any one of the methods disclosed herein is an antibody that binds to, and/or reduces or blocks the function of a protein involved in an immune checkpoint pathway.

In some aspects, the immune checkpoint inhibitor targets cytotoxic T-lymphocyte antigen-4 (CTLA-4). In some aspects, the immune checkpoint inhibitor that targets CTLA-4 is Ipilimumab or tremelimumab (ticilimumab, CP-675,206). In some aspects, the immune checkpoint inhibitor targets PD-1. In some aspects, the immune checkpoint inhibitor that targets PD-1 is nivolumab (ONO-4538/BMS-936558. MDX1106, OPDIVO®), pembrolizumab (MK-3475, KEYTRUDA®), pidilizumab (CT-011), atezolizumab (MPDL3280A), cemiplimab (LIBTAYO™), Spartalizumab (PDR001), Camrelizumab (SHR1210), Sintilimab (1B1308), Tislelizumab (BGB-A317), Toripalimab (JS 001), AMP-224, AMP-514 or Spartalizumab (PDR001). In some aspects, the immune checkpoint inhibitor targets PD-L-1. In some aspects, the immune checkpoint inhibitor that targets PD-L-1 is Avelumab, Atezolizumab, Durvalumab, KN035, CK-301, AUNP12, CA-170, or BMS-986189. In some aspects, the immune checkpoint inhibitor targets T cell immunoglobulin and mucin-domain containing-3 (TIM-3) and/or Lymphocyte Activating 3 (LAG3) proteins. In some aspects, the immune checkpoint inhibitor targeting TIM3 is MBG453: TSR-022; or LY3321367. In some aspects, the immune checkpoint inhibitor targeting LAG3 is IMP321 (Eftilagimod alpha), BMS-986016 (Relatlimab), LAG525 (anti-LAG-3 mAb), REGN3767 (anti-LAG-3 mAb), TSR-033 (anti-LAG-3 mAb), MGD013 (a PD-1/LAG-3 bispecific DART® protein), or FS118 (a LAG-3/PD-L1 bispecific antibody).

In some aspects, the immune checkpoint inhibitor targets indoleamine 2,3-dioxygenase-1 (IDO1). some aspects, the immune checkpoint inhibitor that targets IDO1 is Indoximod (D-1MT; NLG-8189), Navoximod (NLG-919), Epacadostat (INCB024360), BMS-986205, PF-06840003, IOM2983, or RG-70099.

In some aspects, the immune checkpoint inhibitor targets V-domain Ig suppressor of T cell activation (VISTA).

In some aspects, the at least one agent that improves T cell response is a cytokine, such as an inflammatory cytokine. In some aspects, the cytokine is type I IFN or IL-12. In some aspects, the cytokine is a cytokine that shares the common gamma chain receptor. Non-limiting examples of a cytokine that shares the common gamma chain receptor include IL-2, IL-7, IL-15, and IL-21. In some aspects, the at least one agent that improves T cell response is IL-2.

In some aspects, the at least one agent that improves T cell response targets Treg cells. In some aspects, the agent that target Treg cells is an anti-CCR4 antibody, an neuropilin-1 (Nrp-1) inhibitor, or a semaphoring-4a (Sema4a) inhibitor. In some aspects, the at least one agent that improves T cell response is an mTOR inhibitor. Non-limiting examples of an mTOR inhibitor include rapamycin, temsirolimus (CCI-779), everolimus (RAD001), ridaforolimus (AP-23573), sirolimus, dactolisib, BGT226, SF1126. PKI-587, sapanisertib, AZD8055, and AZD2014.

The mode of administration of the immune checkpoint inhibitor is not limited, and may be any mode that is recommended for, or known to be suitable for, the immune checkpoint inhibitor, as described in the art. The mode of administration may vary depending on the particular immune checkpoint inhibitor that is used. In some aspects, the immune checkpoint inhibitor disclosed herein may be administered via a systemic route, such as for example, parenteral routes of administration; a mucosal route: a transdermal route; or directly into a specific tissue. In some aspects, the immune checkpoint inhibitor is administered to a subject in need thereof at a therapeutically effective dose. The therapeutically effective dose depends on factors such as the type of cancer being treated, the age, weight and health of the subject, and route of administration.

The amount of immune checkpoint inhibitor administered to the subject is not limited, and may be any amount as determined by the physician and/or as described or known in the art. In some aspects, the amount of immune checkpoint inhibitor administered to the subject is in the range of about 1 μg/kg to about 50 mg/kg, such as, for example, 5 μg/kg, 10 μg/kg, 50 μg/kg, 100 μg/kg, 500 μg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg, including all values and subranges that lie therebetween. In some aspects, the dose may be administered in a volume of about 0.1 mL to about 1.5 mL, for example, about 0.2 mL, about 0.4 mL, 0.5 mL, about 0.6 mL, about 0.8 mL, about 1 mL, or about 1.2 mL, including all values and subranges that lie therebetween.

In some aspects, the immune checkpoint inhibitor is administered concurrently with the synthetic antibody, scFv, multi-specific ICE, CAR construct or CAR T cell disclosed herein. In some aspects, the immune checkpoint inhibitor is administered prior to the synthetic antibody, scFv, multi-specific ICE, CAR construct or CAR T cell disclosed herein. In some aspects, the immune checkpoint inhibitor is administered after a synthetic antibody, scFv, multi-specific ICE, CAR construct or CAR T cell disclosed herein.

### Generation of Synthetic Antibodies In Vitro and Ex Vivo

In one embodiment, the synthetic antibody, scFv, multi-specific ICE, CAR construct or CAR T cell is generated in vitro or ex vivo. For example, in one embodiment, a nucleic acid encoding a synthetic antibody, scFv, CAR construct or multi-specific ICE can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### Delivery Vehicles

In one embodiment, the present invention provides a composition comprising a delivery vehicle comprising a bispecific anti-CD45 immune cell engaging antibody, scFv, CAR molecule or fragment thereof, or nucleic acid molecule encoding the same, as described herein. In one embodiment, the nucleic acid molecule encoding the bispecific anti-CD45 immune cell engaging antibody scFv, CAR molecule or fragment thereof comprises an mRNA molecule.

Exemplary delivery vehicles include, but are not limited to, microspheres, microparticles, nanoparticles, polymerosomes, liposomes, and micelles. For example, in some embodiments, the delivery vehicle is a lipid nanoparticle loaded with a nucleic acid molecule encoding a bispecific anti-CD45 immune cell engaging antibody of the invention or a fragment thereof. In one embodiment, the nucleic acid molecule encoding the bispecific anti-CD45 immune cell engaging antibody comprises an mRNA molecule.

In some embodiments, the delivery vehicle provides for controlled release, delayed release, or continual release of its loaded cargo. In some embodiments, the delivery vehicle comprises a targeting moiety that targets the delivery vehicle to a treatment site.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 min of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens. 2005, Ann NY Acad Sci 1050:257-265).

In order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

### Methods of Delivery Using Engineered Cells

In various embodiments, the invention relates to a composition comprising an engineered cell. In some embodiments, the engineered cell comprises at least one edited epitope. In some embodiments, the edited cell is a blood cell or a hematopoietic stem cell. The blood cells can be white blood cells or red blood cells. The white blood cells can be immune effector cells. In some embodiments, the white blood cells are T cells, B cells. NK cells, NK-T cells, neutrophils, macrophages, monocytes, dendritic cells. In some embodiments, the method comprises administering a combination of epitope edited T cells and hematopoietic stem cells.

Examples of immune cells that can be engineered include, but are not limited to, T cells, B cells, natural killer (NK) cells, or macrophages. In some embodiments, the immune cell further comprises a chimeric antigen receptor (CAR). Therefore, in some embodiments, the invention relates to the use of CAR T-cells for expression or delivery of a CD45-specific CAR molecule of the invention.

In one embodiment, the present invention provides a method for delivery of a multi-specific immune cell engaging antibody to a target cell providing an engineered immune cell expressing the multi-specific immune cell engaging antibody. In one embodiment, the immune cell is engineered for endogenous secretion of the bispecific multi-specific immune cell engaging antibody. In one embodiment, the immune cell is engineered for surface expression of the multi-specific immune cell engaging antibody. In one embodiment, the engineered cell expressing the multi-specific immune cell engaging antibody comprises at least one epitope modification to one or more epitope targeted by the multi-specific immune cell engaging antibody to reduce binding of the multi-specific immune cell engaging antibody to the engineered cell.

In one embodiment, the present invention provides a method for delivery of a bispecific anti-CD45 immune cell engaging antibody to a target cell providing an engineered immune cell expressing the bispecific anti-CD45 immune cell engaging antibody. In one embodiment, the immune cell is engineered for endogenous secretion of the bispecific anti-CD45 immune cell engaging antibody. In one embodiment, the immune cell is engineered for surface expression of the bispecific anti-CD45 immune cell engaging antibody. In one embodiment, the engineered cell expressing the bispecific anti-CD45 immune cell engaging antibody comprises at least one epitope modification to CD45 to reduce binding of anti-CD45 to the engineered cell.

In various embodiments, the invention relates to compositions for endogenous secretion of a T cell-redirecting bispecific antibody (T-bsAb) by engineered T cells (STAb-T cells), which have been engineered to express the bispecific anti-CD45 immune cell engaging antibody. In various embodiments, the method comprises administering to a subject in need thereof a composition comprising a STAb-T cell, wherein the STAb-T cell has been engineered to express the bispecific anti-CD45 immune cell engaging antibody. In some embodiments, the STAb-T cell further comprises a chimeric antigen receptor (CAR). Therefore, in some embodiments, the invention relates to the use of CAR T-cells for expression or delivery of a bispecific anti-CD45 immune cell engaging antibody.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Development of Bispecific T Cell Engagers for the Immune Targeting of CD45 in Blood Cancers Through Epitope-Editing Gene Therapy

Blood cancers are prevalent in the US, with blood cancers representing 9.8% of new cancer cases. Immunotherapy has been a game-changer in cancers therapeutics, however, this treatment has several limitations including requirement for repeated administration, more limited stability and cost. A further advance on monoclonal technology is the development of bispecific immune cell engagers (BiICE) which combine the specificity of monoclonal antibodies with the cytotoxic potential of T cells. BiICEs have shown promising results in leukemia clinical trials (Viardot et al., 2016, Blood, 127(11):1410-6; Goebeler et al., 2016. J Clin Oncol, 34(10):1104-11), however, this therapy has a limited applicability because it requires continuous intravenous infusion for 4-8 weeks per cycle (Zhu et al., 2016, Clin Pharmacokinet, 55(10):1271-88) and can have limitations for its production. A longer-lived simpler production method for antibody-based products would likely be an important new tool for cancer immunotherapy.

The studies presented herein demonstrate the development of bispecific T-cell engagers (BiTEs) targeting CD45. CD45 is the prototypic member of transmembrane receptor-like protein tyrosine phosphatases (RPTPs) and has essential roles in immune functions. CD45 consists of an extracellular receptor-like region, a short transmembrane segment, and a cytoplasmic region comprising tandem PTP domains.

The BiTE comprises a domain for targeting CD3. The CD3 targeting antibody UCHT1 is humanized. L234A/L235A are LALA mutations that abrogate binding to FcG receptors while sparing any negative effects on FcRn binding. D270A/K322A abrogate complement activity. Expression was done using an expi293 cell expression system. The DNA sequences encoding the BiTE were codon optimized for expression in humans and mice and cloned into an expression vector. pVax1 is an empty plasmid vector negative control, and the subsequent supernatant of nothing being expressed being used as a negative control. CD45-BiTEs bind T cell surface-expressed CD45 and CD3.

MOLM14 is a cell line established from the peripheral blood of a 20-year-old man with acute myeloid leukemia AML FAB M5a at relapse in 1995 after initial myelodysplastic syndrome (MDS, refractory anemia with excess of blasts, RAEB). A luciferase killing assay demonstrated that BC8-BiTEs are highly efficient at killing AML MOLM14 cells. The data were taken at the 24 hour timepoint with the donor T cells at a 5:1 ET ratio.

### Example 2: Anti-CD45/CD3 Bispecific T Cell Engagers Demonstrate High Efficacy Against AML Cells while Sparing Epitope Edited Cells

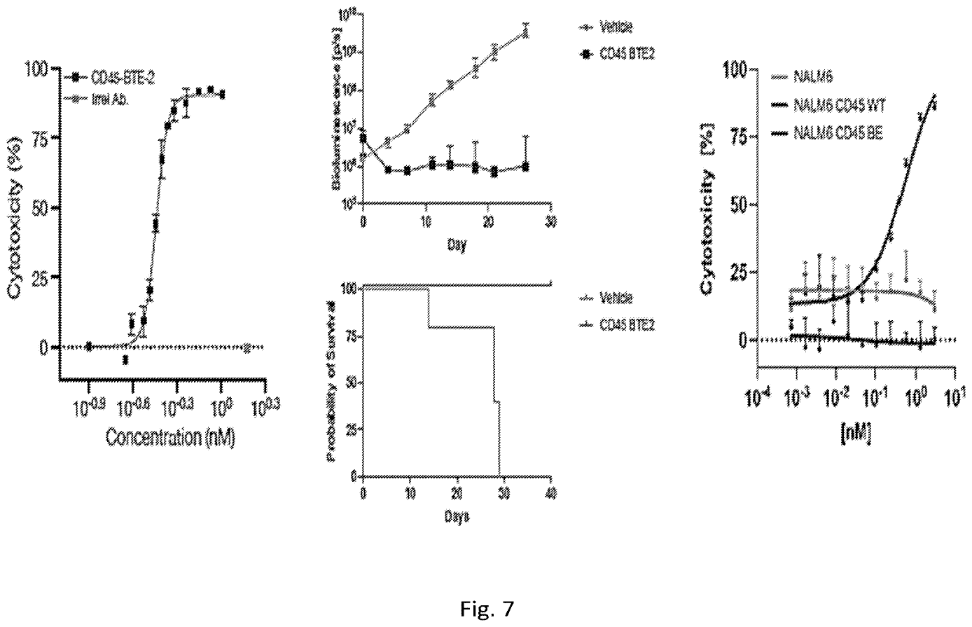

Anti CD45/CD3 bispecific T cell engagers (BiTE) were generated based on clone BC8 (CD45) and UCHL1 (CD3). The BiTE demonstrates dose-dependent cytotoxicity against AML (MOLM14) cells (FIG. 7, left panel) in vitro and in vivo (FIG. 7, middle panel). For the in vivo data, mice were injected with 1×10\hat{ }6 MOLM14 cells and engraftment was confirmed. Subsequently, mice were treated with 10×10\hat{ }6 epitope edited T cells and treated with the anti-CD45/CD3 BiTE at 0.3 mg/kg twice a week and tumor burden was measured by bioluminescent imaging. All mice responded to the BiTE treatment with 4/5 mice clearing their tumor entirely. Importantly. CD45 negative cells (NALM6) expressing the epitope edited CD45 (Y232C) are not being affected in the presences of the anti-CD45/CD3 BiTE. Therefore the epitope edit provides resistance to BTEs and therefore sets the stage to use BiTEs as an alternative to CAR-T cells in combination with HSC epitope editing.

### Example 3: Multi-valent Immune Cell Engagers

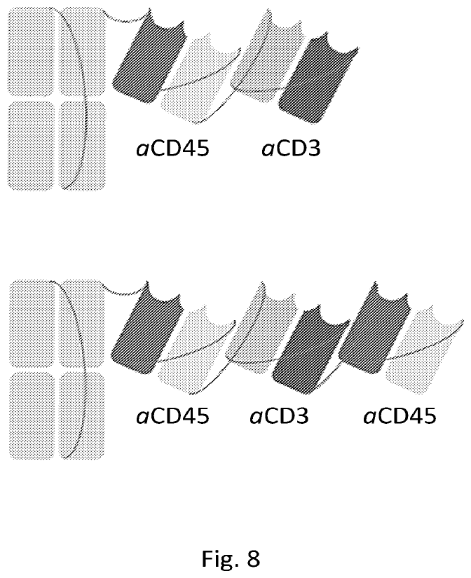

FIG. 8 shows the design of a monovalent and multivalent immune cell engagers. The antibody can be monovalent for CD45, with 1:1 valency ratio for CD45 and CD3. However, the format can also be bivalent for CD45, with two CD45 binding domains.

### Example 4: Sequences

CD45 Variable Heavy Chain
(SEQ ID NO: 1)
QVQLVESGGGLVQPGGSLKLSCAASGFDFSRYWMSWVRQAPGKGLEWIGEINPTSSTINF
TPSLKDKVFISRDNAKNTLYLQMSKVRSEDTALYYCARGNYYRYGDAMDYWGQGTSVTVS
S
CD45 Variable Light Chain
(SEQ ID NO: 2)
DIVLTQSPASLAVSLGQRATISCRASKSVSTSGYSYLHWYQQKPGQPPKLLIYLASNLES
GVPARFSGSGSGTDFTLNIHPVEEEDAATYYCQHSRELPFTFGSGTKLEIK
CD3 Variable Heavy Chain
(SEQ ID NO: 3)
EVQLVESGGGLVQPGGSLRLSCAASGYSFTGYTMNWVRQAPGKGLEWVALINPYKGVSTY
NQKFKDRFTISVDKSKNTAYLQMNSLRAEDTAVYYCARSGYYGDSDWYFDVWGQGTLVTV
SS
CD3 Variable Light Chain
(SEQ ID NO: 4)
DIQMTQSPSSLSASVGDRVTITCRASQDIRNYLNWYQQKPGKAPKLLIYYTSRLESGVPS
RFSGSGSGTDYTLTISSLQPEDFATYYCQQGNTLPWTFGQGTKVEIKSS
CD45 scFv (HL)
SEQ ID NO: 5
QVQLVESGGGLVQPGGSLKLSCAASGFDFSRYWMSWVRQAPGKGLEWIGEINPTSSTINF
TPSLKDKVFISRDNAKNTLYLQMSKVRSEDTALYYCARGNYYRYGDAMDYWGQGTSVTVS
SGGGGSGGGGSGGGGSDIVLTQSPASLAVSLGQRATISCRASKSVSTSGYSYLHWYQQKP
GQPPKLLIYLASNLESGVPARFSGSGSGTDFTLNIHPVEEEDAATYYCQHSRELPFTFGS
GTKLEIK
codon optimized sequence encoding CD45 scFv (HL)
SEQ ID NO: 6
CAGGTGCAGCTGGTGGAGAGCGGCGGCGGCCTGGTGCAGCCTGGCGGCTCCCTGAAGCTG
TCTTGCGCAGCAAGCGGCTTCGACTTTTCTAGGTACTGGATGAGCTGGGTGAGACAGGCA
CCAGGCAAGGGACTGGAGTGGATCGGCGAGATCAACCCAACCAGCTCCACAATCAATTTC
ACCCCCTCCCTGAAGGACAAGGTGTTTATCTCTCGGGATAACGCCAAGAATACCCTGTAT
CTGCAGATGTCCAAGGTGCGCTCTGAGGACACAGCCCTGTACTATTGCGCCAGGGGCAAC
TACTATAGATACGGCGACGCCATGGATTATTGGGGCCAGGGCACCTCCGTGACAGTGTCT
AGCGGCGGCGGCGGCAGCGGCGGCGGCGGCAGCGGCGGCGGCGGCTCCGATATCGTGCTG
ACACAGTCTCCAGCAAGCCTGGCCGTGAGCCTGGGACAGAGGGCAACCATCTCCTGTAGG
GCCTCCAAGTCTGTGAGCACATCCGGCTACAGCTATCTGCACTGGTACCAGCAGAAGCCA
GGACAGCCCCCTAAGCTGCTGATCTATCTGGCAAGCAACCTGGAGTCCGGAGTGCCTGCA
AGGTTCTCTGGAAGCGGATCCGGAACCGACTTTACACTGAATATCCACCCAGTGGAGGAG
GAGGATGCCGCCACCTACTATTGTCAGCACTCTAGGGAGCTGCCCTTCACCTTTGGCAGC
GGCACAAAGCTGGAGATCAAG
CD45 scFv (LH)
SEQ ID NO: 7
DIVLTQSPASLAVSLGQRATISCRASKSVSTSGYSYLHWYQQKPGQPPKLLIYLASNLES
GVPARFSGSGSGTDFTLNIHPVEEEDAATYYCQHSRELPFTFGSGTKLEIKGGGGSGGGG
SGGGGSQVQLVESGGGLVQPGGSLKLSCAASGFDFSRYWMSWVRQAPGKGLEWIGEINPT
SSTINFTPSLKDKVFISRDNAKNTLYLQMSKVRSEDTALYYCARGNYYRYGDAMDYWGQG
TSVTVSSGGGGSEVQLVESGGGLVQPGGSLRLSCAASGYSFTGYTMNWVRQAPGKGLEWV
ALINPYKGVSTYNQKFKDRFTISVDKSKNTAYLQMNSLRAEDTAVYYCARSGYYGDSDWY
FDVWGQGTLVTVSSGGGGSGGGGSGGGGSDIQMTQSPSSLSASVGDRVTITCRASQDIRN
YLNWYQQKPGKAPKLLIYYTSRLESGVPSRFSGSGSGTDYTLTISSLQPEDFATYYCQQG
NTLPWTFGQGTKVEIKSS
codon optimized sequence encoding CD45 scFv (LH)
SEQ ID NO: 8
GACATCGTGCTGACCCAGTCTCCAGCAAGCCTGGCCGTGTCCCTGGGACAGAGGGCAACC
ATCTCTTGCAGGGCCTCCAAGTCTGTGAGCACATCCGGCTACTCCTATCTGCACTGGTAC
CAGCAGAAGCCAGGACAGCCCCCTAAGCTGCTGATCTATCTGGCCTCCAACCTGGAGTCT
GGAGTGCCTGCAAGGTTCTCTGGAAGCGGATCCGGAACCGACTTTACACTGAATATCCAC
CCAGTGGAGGAGGAGGATGCCGCCACATACTATTGCCAGCACTCTAGGGAGCTGCCCTTC
ACCTTTGGCAGCGGCACAAAGCTGGAGATCAAGGGCGGCGGCGGCAGCGGCGGCGGCGGC
AGCGGCGGCGGCGGCTCCCAGGTGCAGCTGGTGGAGAGCGGCGGCGGCCTGGTGCAGCCT
GGCGGCAGCCTGAAGCTGTCCTGTGCAGCATCTGGCTTCGACTTTTCTAGGTACTGGATG
AGCTGGGTGAGACAGGCACCAGGCAAGGGACTGGAGTGGATCGGCGAGATCAACCCAACC
AGCTCCACAATCAATTTCACCCCCAGCCTGAAGGACAAGGTGTTTATCTCCCGGGATAAC
GCCAAGAATACCCTGTATCTGCAGATGAGCAAGGTGCGCTCCGAGGATACAGCCCTGTAC
TATTGTGCCAGGGGCAACTACTATAGATACGGCGACGCCATGGATTATTGGGGCCAGGGC
ACCTCCGTGACAGTGTCTAGC
CD3 scFv (HL)
SEQ ID NO: 9
EVQLVESGGGLVQPGGSLRLSCAASGYSFTGYTMNWVRQAPGKGLEWVALINPYKGVSTY
NQKFKDRFTISVDKSKNTAYLQMNSLRAEDTAVYYCARSGYYGDSDWYFDVWGQGTLVTV
SSGGGGSGGGGSGGGGSDIQMTQSPSSLSASVGDRVTITCRASQDIRNYLNWYQQKPGKA
PKLLIYYTSRLESGVPSRFSGSGSGTDYTLTISSLQPEDFATYYCQQGNTLPWTFGQGTK
VEIKSS
sequence encoding CD3 scFv (HL)
SEQ ID NO: 10
GAGGTGCAGCTGGTGGAGAGCGGCGGCGGCCTGGTGCAGCCAGGCGGCAGCCTGAGGCTG
TCCTGTGCAGCAAGCGGATACTCCTTCACCGGCTATACAATGAATTGGGTGAGGCAGGCC
CCTGGCAAGGGCCTGGAATGGGTGGCCCTGATCAACCCCTACAAGGGCGTGTCCACCTAT
AATCAGAAGTTCAAGGACCGCTTTACCATCTCTGTGGATAAGAGCAAGAACACAGCCTAC
CTGCAGATGAATAGCCTGAGAGCCGAGGACACAGCCGTGTACTATTGCGCACGGAGCGGA
TACTATGGCGACTCCGATTGGTATTTTGACGTGTGGGGCCAGGGCACCCTGGTGACAGTG
AGCAGCGGCGGCGGCGGCAGCGGCGGCGGCGGCAGCGGCGGCGGCGGCTCTGACATCCAG
ATGACCCAGTCCCCATCTAGCCTGTCTGCCAGCGTCGGCGACAGGGTGACCATCACATGT
CGCGCCTCTCAGGATATCAGGAACTACCTGAATTGGTATCAGCAGAAGCCCGGCAAGGCC
CCTAAGCTGCTGATCTACTATACATCCAGGCTGGAGTCTGGAGTGCCAAGCAGGTTCTCC
GGATCTGGAAGCGGAACCGACTACACCCTGACAATCTCCTCTCTGCAGCCCGAGGATTTC
GCCACATACTATTGTCAGCAGGGCAATACCCTGCCTTGGACATTTGGCCAGGGCACCAAG
GTGGAGATCAAGAGCTCC
BC8-LHHL-BITE
BC8-LHHL-BiTE, no his tag
SEQ ID NO: 11
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKENWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSDIVLTQSPASLAVSLGQRATISCRASKS
VSTSGYSYLHWYQQKPGQPPKLLIYLASNLESGVPARFSGSGSGTDFTLNIHPVEEEDAA
TYYCQHSRELPFTFGSGTKLEIKGGGGSGGGGSGGGGSQVQLVESGGGLVQPGGSLKLSC
AASGFDFSRYWMSWVRQAPGKGLEWIGEINPTSSTINFTPSLKDKVFISRDNAKNTLYLQ
MSKVRSEDTALYYCARGNYYRYGDAMDYWGQGTSVTVSSGGGGSEVQLVESGGGLVQPGG
SLRLSCAASGYSFTGYTMNWVRQAPGKGLEWVALINPYKGVSTYNQKFKDRFTISVDKSK
NTAYLQMNSLRAEDTAVYYCARSGYYGDSDWYFDVWGQGTLVTVSSGGGGSGGGGSGGGG
SDIQMTQSPSSLSASVGDRVTITCRASQDIRNYLNWYQQKPGKAPKLLIYYTSRLESGVP
SRFSGSGSGTDYTLTISSLQPEDFATYYCQQGNTLPWTFGQGTKVEIKSS
BC8-LHHL-BiTE, with his tag
SEQ ID NO: 12
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSDIVLTQSPASLAVSLGQRATISCRASKS
VSTSGYSYLHWYQQKPGQPPKLLIYLASNLESGVPARFSGSGSGTDFTLNIHPVEEEDAA
TYYCQHSRELPFTFGSGTKLEIKGGGGSGGGGSGGGGSQVQLVESGGGLVQPGGSLKLSC
AASGFDFSRYWMSWVRQAPGKGLEWIGEINPTSSTINFTPSLKDKVFISRDNAKNTLYLQ
MSKVRSEDTALYYCARGNYYRYGDAMDYWGQGTSVTVSSGGGGSEVQLVESGGGLVQPGG
SLRLSCAASGYSFTGYTMNWVRQAPGKGLEWVALINPYKGVSTYNQKFKDRFTISVDKSK
NTAYLQMNSLRAEDTAVYYCARSGYYGDSDWYFDVWGQGTLVTVSSGGGGSGGGGSGGGG
SDIQMTQSPSSLSASVGDRVTITCRASQDIRNYLNWYQQKPGKAPKLLIYYTSRLESGVP
SRFSGSGSGTDYTLTISSLQPEDFATYYCQQGNTLPWTFGQGTKVEIKSSHHHHHH
BC8-HLHL-BiTE, no his tag
SEQ ID NO: 13
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSQVQLVESGGGLVQPGGSLKLSCAASGFD
FSRYWMSWVRQAPGKGLEWIGEINPTSSTINFTPSLKDKVFISRDNAKNTLYLQMSKVRS
EDTALYYCARGNYYRYGDAMDYWGQGTSVTVSSGGGGSGGGGSGGGGSDIVLTQSPASLA
VSLGQRATISCRASKSVSTSGYSYLHWYQQKPGQPPKLLIYLASNLESGVPARFSGSGSG
TDFTLNIHPVEEEDAATYYCQHSRELPFTFGSGTKLEIKGGGGSEVQLVESGGGLVQPGG
SLRLSCAASGYSFTGYTMNWVRQAPGKGLEWVALINPYKGVSTYNQKFKDRFTISVDKSK
NTAYLQMNSLRAEDTAVYYCARSGYYGDSDWYFDVWGQGTLVTVSSGGGGGGGGSGGGGS
DIQMTQSPSSLSASVGDRVTITCRASQDIRNYLNWYQQKPGKAPKLLIYYTSRLESGVPS
RFSGSGSGTDYTLTISSLQPEDFATYYCQQGNTLPWTFGQGTKVEIKSS
BC8-HLHL-BITE, with his tag
SEQ ID NO: 14
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGGGGGSQVQLVESGGGLVQPGGSLKLSCAASGFDF
SRYWMSWVRQAPGKGLEWIGEINPTSSTINFTPSLKDKVFISRDNAKNTLYLQMSKVRSE
DTALYYCARGNYYRYGDAMDYWGQGTSVTVSSGGGGSGGGGSGGGGSDIVLTQSPASLAV
SLGQRATISCRASKSVSTSGYSYLHWYQQKPGQPPKLLIYLASNLESGVPARFSGSGSGT
DFTLNIHPVEEEDAATYYCQHSRELPFTFGSGTKLEIKGGGGSEVQLVESGGGLVQPGGS
LRLSCAASGYSFTGYTMNWVRQAPGKGLEWVALINPYKGVSTYNQKFKDRFTISVDKSKN
TAYLQMNSLRAEDTAVYYCARSGYYGDSDWYFDVWGQGTLVTVSSGGGGSGGGGSGGGGS
DIQMTQSPSSLSASVGDRVTITCRASQDIRNYLNWYQQKPGKAPKLLIYYTSRLESGVPS
RFSGSGSGTDYTLTISSLQPEDFATYYCQQGNTLPWTFGQGTKVEIKSSHHHHHH
Full RNA for CD45-BiTE-1 (LHHL) (containing additional N-
terminal and C-terminal sequences which facilitate enhanced
translation)
SEQ ID NO: 15
GCTGCTTCGCGATGTACGGGCCAGATATACGCGTTGACATTGATTATTGACTACTTATTA
ATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAAT
AATGACGTATGTTCCCATAGTAACGCCAATAGGGACTTTCCATTGACGTCAATGGGTGGA
GTATTTACGGTAAACTGCCCACTTGGCAGTACATCAAGTGTATCATATGCCAAGTACGCC
CCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAG
TCTCCACCCCATTGACGTCAATGGGAGTTTGTTTTGGCACCAAAATCAACGGGACTTTCC
AAAATGTCGTAACAACTCCGCCCCATTGACGCAAATGGGCGGTAGGCGTGTACGGTGGGA
GGTCTATATAAGCAGAGCTCTCTGGCTAACTAGAGAACCCACTGCTTACTGGCTTATCGA
AATTAATACGACTCACTATAGGGAGACCCAAGCTGGCTAGCGTTTAAACTTAAGCTTGGT
ACCGAGCTCGGATCCACTAGTCCAGTGTGGTGGAATTCGCCGCCACCATGGACTGGACCT
GGATCCTGTTCCTGGTGGCTGCCGCCACCAGGGTGCACAGCGACAAGACCCACACATGCC
CTCCATGTCCAGCACCAGAGGCAGCCGGCGGACCTAGCGTGTTCCTGTTTCCCCCTAAGC
CAAAGGATACCCTGATGATCTCTCGGACCCCTGAGGTGACATGCGTGGTGGTGGACGTGA
GCCACGAGGCACCAGAGGTGAAGTTCAACTGGTACGTGGATGGCGTGGAGGTGCACAATG
CCAAGACAAAGCCTTGCGAGGAGCAGTACGGCTCCACCTATCGCTGCGTGAGCGTGCTGA
CAGTGCTGCACCAGGACTGGCTGAACGGCAAGGAGTATAAGTGCGCCGTGTCCAATAAGG
CCCTGCCTGCCCCAATCGAGAAGACCATCTCTAAGGCCAAGGGCCAGCCTAGGGAGCCAC
AGGTGTACACACTGCCACCCTCCAGAGAGGAGATGACCAAGAACCAGGTGTCTCTGACAT
GTCTGGTGAAGGGCTTCTATCCCAGCGACATCGCCGTGGAGTGGGAGTCCAATGGCCAGC
CTGAGAACAATTACAAGACCACACCTCCAGTGCTGGACTCTGATGGCAGCTTCTTTCTGT
ATTCTAAGCTGACCGTGGATAAGAGCCGGTGGCAGCAGGGCAACGTGTTTAGCTGCTCCG
TGATGCACGAGGCCCTGCACAATCACTACACACAGAAGTCTCTGAGCCTGTCCCCAGGCA
AGGGCGGCGGCGGCAGCGGCGGCGGCGGCAGCGGCGGCGGCGGCAGCGGCGGCGGCGGCA
GCGGCGGCGGCGGCAGCGGCGGCGGCGGCTCGGATAAAACCCACACATGCCCTCCCTGTC
CAGCACCTGAGGCAGCCGGCGGACCAAGCGTGTTCCTGTTTCCACCCAAGCCTAAAGACA
CCCTGATGATCAGCAGGACCCCCGAAGTCACCTGCGTGGTCGTGGACGTGTCCCACGAGG
CACCTGAAGTCAAGTTCAACTGGTACGTGGACGGAGTCGAAGTCCATAACGCCAAGACAA
AGCCCTGTGAAGAGCAGTACGGCAGCACCTATAGATGCGTGAGCGTGCTGACAGTGCTGC
ATCAGGATTGGCTGAATGGCAAGGAATACAAGTGCGCCGTGTCTAATAAGGCCCTGCCAG
CCCCCATCGAGAAGACCATCTCCAAGGCAAAGGGACAGCCAAGGGAGCCTCAGGTGTACA
CACTGCCTCCAAGCCGCGAAGAAATGACTAAAAACCAGGTGTCCCTGACCTGCCTGGTCA
AAGGCTTCTATCCATCTGATATTGCCGTGGAGTGGGAGAGCAATGGCCAGCCCGAAAATA
ATTACAAGACCACACCCCCTGTGCTGGACTCCGATGGCTCTTTCTTTCTGTATTCCAAAC
TGACTGTGGATAAGTCTCGCTGGCAGCAGGGAAACGTGTTTTCTTGTAGCGTGATGCATG
AGGCCCTGCATAACCACTACACACAGAAGTCCCTGTCTCTGAGCCCTGGCAAGGGCGGCG
GCGGCAGCGGCGGCGGCGGCTCTGACATCGTGCTGACCCAGTCTCCAGCAAGCCTGGCCG
TGTCCCTGGGACAGAGGGCAACCATCTCTTGCAGGGCCTCCAAGTCTGTGAGCACATCCG
GCTACTCCTATCTGCACTGGTACCAGCAGAAGCCAGGACAGCCCCCTAAGCTGCTGATCT
ATCTGGCCTCCAACCTGGAGTCTGGAGTGCCTGCAAGGTTCTCTGGAAGCGGATCCGGAA
CCGACTTTACACTGAATATCCACCCAGTGGAGGAGGAGGATGCCGCCACATACTATTGCC
AGCACTCTAGGGAGCTGCCCTTCACCTTTGGCAGCGGCACAAAGCTGGAGATCAAGGGCG
GCGGCGGCAGCGGCGGCGGCGGCAGCGGCGGCGGCGGCTCCCAGGTGCAGCTGGTGGAGA
GCGGCGGCGGCCTGGTGCAGCCTGGCGGCAGCCTGAAGCTGTCCTGTGCAGCATCTGGCT
TCGACTTTTCTAGGTACTGGATGAGCTGGGTGAGACAGGCACCAGGCAAGGGACTGGAGT
GGATCGGCGAGATCAACCCAACCAGCTCCACAATCAATTTCACCCCCAGCCTGAAGGACA
AGGTGTTTATCTCCCGGGATAACGCCAAGAATACCCTGTATCTGCAGATGAGCAAGGTGC
GCTCCGAGGATACAGCCCTGTACTATTGTGCCAGGGGCAACTACTATAGATACGGCGACG
CCATGGATTATTGGGGCCAGGGCACCTCCGTGACAGTGTCTAGCGGCGGCGGCGGCTCGG
AGGTGCAGCTGGTGGAGAGCGGCGGCGGCCTGGTGCAGCCAGGCGGCAGCCTGAGGCTGT
CCTGTGCAGCAAGCGGATACTCCTTCACCGGCTATACAATGAATTGGGTGAGGCAGGCCC
CTGGCAAGGGCCTGGAATGGGTGGCCCTGATCAACCCCTACAAGGGCGTGTCCACCTATA
ATCAGAAGTTCAAGGACCGCTTTACCATCTCTGTGGATAAGAGCAAGAACACAGCCTACC
TGCAGATGAATAGCCTGAGAGCCGAGGACACAGCCGTGTACTATTGCGCACGGAGCGGAT
ACTATGGCGACTCCGATTGGTATTTTGACGTGTGGGGCCAGGGCACCCTGGTGACAGTGA
GCAGCGGCGGCGGCGGCAGCGGCGGCGGCGGCAGCGGCGGCGGCGGCTCTGACATCCAGA
TGACCCAGTCCCCATCTAGCCTGTCTGCCAGCGTCGGCGACAGGGTGACCATCACATGTC
GCGCCTCTCAGGATATCAGGAACTACCTGAATTGGTATCAGCAGAAGCCCGGCAAGGCCC
CTAAGCTGCTGATCTACTATACATCCAGGCTGGAGTCTGGAGTGCCAAGCAGGTTCTCCG
GATCTGGAAGCGGAACCGACTACACCCTGACAATCTCCTCTCTGCAGCCCGAGGATTTCG
CCACATACTATTGTCAGCAGGGCAATACCCTGCCTTGGACATTTGGCCAGGGCACCAAGG
TGGAGATCAAGAGCTCCTGATAAGCGGCCGCTCGAGTCTAGAGGGCCCGTTTAAACCCGC
TGATCAGCCTCGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGACAGC
AAGGGGGAGGATTGGGAAGACAATAGCAGGCATGCTGGGGATGCGGTGGGCTCTATGGCT
TCTACTGGGCGGTTTTATGGACAGCAAGCGAACCGGAATTGCCAGCTGGGGCGCCCTCTG
GTAAGGTTGGGAAGCCCTGCAAAGTAAACTGGATGGCTTTCTTGCCGCCAAGGATCTGAT
GGCGCAGGGGATCAAGCTCTGATCAAGAGACAGGATGAGGATCGTTTCGCATGATTGAAC
AAGATGGATTGCACGCAGGTTCTCCGGCCGCTTGGGTGGAGAGGCTATTCGGCTATGACT
GGGCACAACAGACAATCGGCTGCTCTGATGCCGCCGTGTTCCGGCTGTCAGCGCAGGGGC
GCCCGGTTCTTTTTGTCAAGACCGACCTGTCCGGTGCCCCGAATGAACTGCAAGACGAGG
CAGCGCGGCTATCGTGGCTGGCCACGACGGGCGTTCCTTGCGCAGCTGTGCTCGACGTTG
TCACTGAAGCGGGAAGGGACTGGCTGCTATTGGGCGAAGTGCCGGGGCAGGATCTCCTGT
CATCTCACCTTGCCCTGCCGAGAAAGTATCCATCATGGCTGATGCAATGCGGCGGCTGC
ATACGCTTGATCCGGCTACCTGCCCATTCGACCACCAAGCGAAACATCGCATCGAGCGAG
CACGTACTCGGATGGAAGCCGGTCTTGTCGATCAGGATGATCTGGACGAAGAGCATCAGG
GGCTCGCGCCAGCCGAACTGTTCGCCAGGCTCAAGGCGAGCATGCCCGACGGCGAGGATC
TCGTCGTGACCCATGGCGATGCCTGCTTGCCGAATATCATGGTGGAAAATGGCCGCTTTT
CTGGATTCATCGACTGTGGCCGGCTGGGTGTGGCGGACCGCTATCAGGACATAGCGTTGG
CTACCCGTGATATTGCTGAAGAGCTTGGCGGCGAATGGGCTGACCGCTTCCTCGTGCTTT
ACGGTATCGCCGCTCCCGATTCGCAGCGCATCGCCTTCTATCGCCTTCTTGACGAGTTCT
TCTGAATTATTAACGCTTACAATTTCCTGATGCGGTATTTTCTCCTTACGCATCTGTGCG
GTATTTCACACCGCATCAGGTGGCACTTTTCGGGGAAATGTGCGCGGAACCCCTATTTGT
TTATTTTTCTAAATACATTCAAATATGTATCCGCTCATGAGACAATAACCCTGATAAATG
CTTCAATAATAGCACGTGCTAAAACTTCATTTTTAATTTAAAAGGATCTAGGTGAAGATC
CTTTTTGATAATCTCATGACCAAAATCCCTTAACGTGAGTTTTCGTTCCACTGAGCGTCA
GACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACCGGG
TTGGACTCAAGACGATAGTTACCGGATAAGGCGCAGCGGTCGGGCTGAACGGGGGGTTCG
TGCACACAGCCCAGCTTGGAGCGAACCACCTACACCGAACTGAGATACCTACAGCGTGAG
CTATGAGAAAGCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCCTTTTGC
TGGCCTTTTGCTCACATGTTCTT
RNA coding sequence for CD45-BiTE-1 (LHHL)
SEQ ID NO: 16
ATGGACTGGACCTGGATCCTGTTCCTGGTGGCTGCCGCCACCAGGGTGCACAGCGACAAG
ACCCACACATGCCCTCCATGTCCAGCACCAGAGGCAGCCGGCGGACCTAGCGTGTTCCTG
TTTCCCCCTAAGCCAAAGGATACCCTGATGATCTCTCGGACCCCTGAGGTGACATGCGTG
GTGGTGGACGTGAGCCACGAGGCACCAGAGGTGAAGTTCAACTGGTACGTGGATGGCGTG
GAGGTGCACAATGCCAAGACAAAGCCTTGCGAGGAGCAGTACGGCTCCACCTATCGCTGC
GTGAGCGTGCTGACAGTGCTGCACCAGGACTGGCTGAACGGCAAGGAGTATAAGTGCGCC
GTGTCCAATAAGGCCCTGCCTGCCCCAATCGAGAAGACCATCTCTAAGGCCAAGGGCCAG
CCTAGGGAGCCACAGGTGTACACACTGCCACCCTCCAGAGAGGAGATGACCAAGAACCAG
GTGTCTCTGACATGTCTGGTGAAGGGCTTCTATCCCAGCGACATCGCCGTGGAGTGGGAG
TCCAATGGCCAGCCTGAGAACAATTACAAGACCACACCTCCAGTGCTGGACTCTGATGGC
AGCTTCTTTCTGTATTCTAAGCTGACCGTGGATAAGAGCCGGTGGCAGCAGGGCAACGTG
TTTAGCTGCTCCGTGATGCACGAGGCCCTGCACAATCACTACACACAGAAGTCTCTGAGC
CTGTCCCCAGGCAAGGGCGGCGGCGGCAGCGGCGGCGGCGGCAGCGGCGGCGGCGGCAGC
GGCGGCGGCGGCAGCGGCGGCGGCGGCAGCGGCGGCGGCGGCTCGGATAAAACCCACACA
TGCCCTCCCTGTCCAGCACCTGAGGCAGCCGGCGGACCAAGCGTGTTCCTGTTTCCACCC
AAGCCTAAAGACACCCTGATGATCAGCAGGACCCCCGAAGTCACCTGCGTGGTCGTGGAC
GTGTCCCACGAGGCACCTGAAGTCAAGTTCAACTGGTACGTGGACGGAGTCGAAGTCCAT
AACGCCAAGACAAAGCCCTGTGAAGAGCAGTACGGCAGCACCTATAGATGCGTGAGCGTG
CTGACAGTGCTGCATCAGGATTGGCTGAATGGCAAGGAATACAAGTGCGCCGTGTCTAAT
AAGGCCCTGCCAGCCCCCATCGAGAAGACCATCTCCAAGGCAAAGGGACAGCCAAGGGAG
CCTCAGGTGTACACACTGCCTCCAAGCCGCGAAGAAATGACTAAAAACCAGGTGTCCCTG
ACCTGCCTGGTCAAAGGCTTCTATCCATCTGATATTGCCGTGGAGTGGGAGAGCAATGGC
CAGCCCGAAAATAATTACAAGACCACACCCCCTGTGCTGGACTCCGATGGCTCTTTCTTT
CTGTATTCCAAACTGACTGTGGATAAGTCTCGCTGGCAGCAGGGAAACGTGTTTTCTTGT
AGCGTGATGCATGAGGCCCTGCATAACCACTACACACAGAAGTCCCTGTCTCTGAGCCCT
GGCAAGGGCGGCGGCGGCAGCGGCGGCGGCGGCTCTGACATCGTGCTGACCCAGTCTCCA
GCAAGCCTGGCCGTGTCCCTGGGACAGAGGGCAACCATCTCTTGCAGGGCCTCCAAGTCT
GTGAGCACATCCGGCTACTCCTATCTGCACTGGTACCAGCAGAAGCCAGGACAGCCCCCT
AAGCTGCTGATCTATCTGGCCTCCAACCTGGAGTCTGGAGTGCCTGCAAGGTTCTCTGGA
AGCGGATCCGGAACCGACTTTACACTGAATATCCACCCAGTGGAGGAGGAGGATGCCGCC
ACATACTATTGCCAGCACTCTAGGGAGCTGCCCTTCACCTTTGGCAGCGGCACAAAGCTG
GAGATCAAGGGCGGCGGCGGCAGCGGCGGCGGCGGCAGCGGCGGCGGCGGCTCCCAGGTG
CAGCTGGTGGAGAGCGGCGGCGGCCTGGTGCAGCCTGGCGGCAGCCTGAAGCTGTCCTGT
GCAGCATCTGGCTTCGACTTTTCTAGGTACTGGATGAGCTGGGTGAGACAGGCACCAGGC
AAGGGACTGGAGTGGATCGGCGAGATCAACCCAACCAGCTCCACAATCAATTTCACCCCC
AGCCTGAAGGACAAGGTGTTTATCTCCCGGGATAACGCCAAGAATACCCTGTATCTGCAG
ATGAGCAAGGTGCGCTCCGAGGATACAGCCCTGTACTATTGTGCCAGGGGCAACTACTAT
AGATACGGCGACGCCATGGATTATTGGGGCCAGGGCACCTCCGTGACAGTGTCLAGCGGC
GGCGGCGGCTCGGAGGTGCAGCTGGTGGAGAGCGGCGGCGGCCTGGTGCAGCCAGGCGGC
AGCCTGAGGCTGTCCTGTGCAGCAAGCGGATACTCCTTCACCGGCTATACAATGAATTGG
GTGAGGCAGGCCCCTGGCAAGGGCCTGGAATGGGTGGCCCTGATCAACCCCTACAAGGGC
GTGTCCACCTATAATCAGAAGTTCAAGGACCGCTTTACCATCTCTGTGGATAAGAGCAAG
AACACAGCCTACCTGCAGATGAATAGCCTGAGAGCCGAGGACACAGCCGTGTACTATTGC
GCACGGAGCGGATACTATGGCGACTCCGATTGGTATTTTGACGTGTGGGGCCAGGGCACC
CTGGTGACAGTGAGCAGCGGCGGCGGCGGCAGCGGCGGCGGCGGCAGCGGCGGCGGCGGC
TCTGACATCCAGATGACCCAGTCCCCATCTAGCCTGTCTGCCAGCGTCGGCGACAGGGTG
ACCATCACATGTCGCGCCTCTCAGGATATCAGGAACTACCTGAATTGGTATCAGCAGAAG
CCCGGCAAGGCCCCTAAGCTGCTGATCTACTATACATCCAGGCTGGAGTCTGGAGTGCCA
AGCAGGTTCTCCGGATCTGGAAGCGGAACCGACTACACCCTGACAATCTCCTCTCTGCAG
CCCGAGGATTTCGCCACATACTATTGTCAGCAGGGCAATACCCTGCCTTGGACATTTGGC
CAGGGCACCAAGGTGGAGATCAAGAGCTCCTGATAA
Full RNA for CD45-BiTE-2 (HLHL)
(containing additional N-terminal and C-terminal sequences
which facilitate enhanced translation)
SEQ ID NO: 17
GCTGCTTCGCGATGTACGGGCCAGATATACGCGTTGACATTGATTATTGACT
AGTTATTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAA
TGGGTGGAGTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCTATTACC
ATGGTGATGCGGTTTTGGCAGTACATCAATGGGCGTGGATAGCGGTTTGACTCACGGGGA
TTTCCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCGTGTA
CGGTGGGAGGTCTATATAAGCAGAGCTCTCTGGCTAACTAGAGAACCCACTGCTTACTGG
CTTATCGAAATTAATACGACTCACTATAGGGAGACCCAAGCTGGCTAGCGTTTAAACTTA
AGCTTGGTACCGAGCTCGGATCCACTAGTCCAGTGTGGTGGAATTCGCCGCCACCATGGA
CTGGACCTGGATCCTGTTCCTGGTGGCTGCCGCCACCAGGGTGCACAGCGACAAGACCCA
CACATGCCCTCCATGTCCAGCACCAGAGGCAGCCGGCGGACCTAGCGTGTTCCTGTTTCC
CCCTAAGCCAAAGGATACCCTGATGATCTCTCGGACCCCTGAGGTGACATGCGTGGTGGT
GGACGTGAGCCACGAGGCACCAGAGGTGAAGTTCAACTGGTACGTGGATGGCGTGGAGGT
GCACAATGCCAAGACAAAGCCTTGCGAGGAGCAGTACGGCTCCACCTATCGCTGCGTGAG
CGTGCTGACAGTGCTGCACCAGGACTGGCTGAACGGCAAGGAGTATAAGTGCGCCGTGTC
CAATAAGGCCCTGCCTGCCCCAATCGAGAAGACCATCTCTAAGGCCAAGGGCCAGCCTAG
GGAGCCACAGGTGTACACACTGCCACCCTCCAGAGAGGAGATGACCAAGAACCAGGTGTC
TCTGACATGTCTGGTGAAGGGCTTCTATCCCAGCGACATCGCCGTGGAGTGGGAGTCCAA
TGGCCAGCCTGAGAACAATTACAAGACCACACCTCCAGTGCTGGACTCTGATGGCAGCTT
CTTTCTGTATTCTAAGCTGACCGTGGATAAGAGCCGGTGGCAGCAGGGCAACGTGTTTAG
CTGCTCCGTGATGCACGAGGCCCTGCACAATCACTACACACAGAAGTCTCTGAGCCTGTC
CCCAGGCAAGGGCGGCGGCGGCAGCGGCGGCGGCGGCAGCGGCGGCGGCGGCAGCGGCGG
CGGCGGCAGCGGCGGCGGCGGCAGCGGCGGCGGCGGCTCGGATAAAACCCACACATGCCC
TCCCTGTCCAGCACCTGAGGCAGCCGGCGGACCAAGCGTGTTCCTGTTTCCACCCAAGCC
TAAAGACACCCTGATGATCAGCAGGACCCCCGAAGTCACCTGCGTGGTCGTGGACGTGTC
CCACGAGGCACCTGAAGTCAAGTTCAACTGGTACGTGGACGGAGTCGAAGTCCATAACGC
CAAGACAAAGCCCTGTGAAGAGCAGTACGGCAGCACCTATAGATGCGTGAGCGTGCTGAC
AGTGCTGCATCAGGATTGGCTGAATGGCAAGGAATACAAGTGCGCCGTGTCTAATAAGGC
CCTGCCAGCCCCCATCGAGAAGACCATCTCCAAGGCAAAGGGACAGCCAAGGGAGCCTCA
GGTGTACACACTGCCTCCAAGCCGCGAAGAAATGACTAAAAACCAGGTGTCCCTGACCTG
CCTGGTCAAAGGCTTCTATCCATCTGATATTGCCGTGGAGTGGGAGAGCAATGGCCAGCC
CGAAAATAATTACAAGACCACACCCCCTGTGCTGGACTCCGATGGCTCTTTCTTTCTGTA
TTCCAAACTGACTGTGGATAAGTCTCGCTGGCAGCAGGGAAACGTGTTTTCTTGTAGCGT
GATGCATGAGGCCCTGCATAACCACTACACACAGAAGTCCCTGTCTCTGAGCCCTGGCAA
GGGCGGCGGCGGCAGCGGCGGCGGCGGCTCTGACATCGTGCTGACCCAGTCTCCAGCAAG
CCTGGCCGTGTCCCTGGGACAGAGGGCAACCATCTCTTGCAGGGCCTCCAAGTCTGTGAG
CACATCCGGCTACTCCTATCTGCACTGGTACCAGCAGAAGCCAGGACAGCCCCCTAAGCT
GCTGATCTATCTGGCCTCCAACCTGGAGTCTGGAGTGCCTGCAAGGTTCTCTGGAAGCGG
ATCCGGAACCGACTTTACACTGAATATCCACCCAGTGGAGGAGGAGGATGCCGCCACATA
CTATTGCCAGCACTCTAGGGAGCTGCCCTTCACCTTTGGCAGCGGCACAAAGCTGGAGAT
CAAGGGCGGCGGCGGCAGCGGCGGCGGCGGCAGCGGCGGCGGCGGCTCCCAGGTGCAGCT
GGTGGAGAGCGGCGGCGGCCTGGTGCAGCCTGGCGGCAGCCTGAAGCTGTCCTGTGCAGC
ATCTGGCTTCGACTTTTCTAGGTACTGGATGAGCTGGGTGAGACAGGCACCAGGCAAGGG
ACTGGAGTGGATCGGCGAGATCAACCCAACCAGCTCCACAATCAATTTCACCCCCAGCCT
GAAGGACAAGGTGTTTATCTCCCGGGATAACGCCAAGAATACCCTGTATCTGCAGATGAG
CAAGGTGCGCTCCGAGGATACAGCCCTGTACTATTGTGCCAGGGGCAACTACTATAGATA
CGGCGACGCCATGGATTATTGGGGCCAGGGCACCTCCGTGACAGTGTCTAGCGGCGGCGG
CGGCTCGGAGGTGCAGCTGGTGGAGAGCGGCGGCGGCCTGGTGCAGCCAGGCGGCAGCCT
GAGGCTGTCCTGTGCAGCAAGCGGATACTCCTTCACCGGCTATACAATGAATTGGGTGAG
GCAGGCCCCTGGCAAGGGCCTGGAATGGGTGGCCCTGATCAACCCCTACAAGGGCGTGTC
CACCTATAATCAGAAGTTCAAGGACCGCTTTACCATCTCTGTGGATAAGAGCAAGAACAC
AGCCTACCTGCAGATGAATAGCCTGAGAGCCGAGGACACAGCCGTGTACTATTGCGCACG
GAGCGGATACTATGGCGACTCCGATTGGTATTTTGACGTGTGGGGCCAGGGCACCCTGGT
GACAGTGAGCAGCGGCGGCGGCGGCAGCGGCGGCGGCGGCAGCGGCGGCGGCGGCTCTGA
CATCCAGATGACCCAGTCCCCATCTAGCCTGTCTGCCAGCGTCGGCGACAGGGTGACCAT
CACATGTCGCGCCTCTCAGGATATCAGGAACTACCTGAATTGGTATCAGCAGAAGCCCGG
CAAGGCCCCTAAGCTGCTGATCTACTATACATCCAGGCTGGAGTCTGGAGTGCCAAGCAG
GTTCTCCGGATCTGGAAGCGGAACCGACTACACCCTGACAATCTCCTCTCTGCAGCCCGA
GGATTTCGCCACATACTATTGTCAGCAGGGCAATACCCTGCCTTGGACATTTGGCCAGGG
CACCAAGGTGGAGATCAAGAGCTCCTGATAAGCGGCCGCTCGAGTCTAGAGGGCCCGTTT
AAACCCGCTGATCAGCCTCGACTGTGCCTTCTAGTTGCCAGCCATCTGTTGTTTGCCCCT
CCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCT
CTATGGCTTCTACTGGGCGGTTTTATGGACAGCAAGCGAACCGGAATTGCCAGCTGGGGC
GCCCTCTGGTAAGGTTGGGAAGCCCTGCAAAGTAAACTGGATGGCTTTCTTGCCGCCAAG
GATCTGATGGCGCAGGGGATCAAGCTCTGATCAAGAGACAGGATGAGGATCGTTTCGCAT
GATTGAACAAGATGGATTGCACCCAGGTTCTCCCCCCGCTTGGGTGGAGAGGCTATTCGG
CTATCACTGGGCACAACAGACAATCGGCTGCTCTGATGCCGCCGTGTTCCGGCTGTCAGC
GCAGGGGCGCCCGGTTCTTTTTGTCAAGACCGACCTGTCCGGTGCCCPGAATGAACTGCA
AGACGAGGCAGCGCGGCTATCGTGGCTGGCCACGACGGGCGTTCCTTGCGCAGCTGTGCT
CGACGTTGTCACTGAAGCGGGAAGGGACTGGCTGCTATTGGGCGAAGTGCCGGGGCAGGA
TCTCCTGTCATCTCACCTTGCTCCTGCCGAGAAAGTATCCATCATGGCTGATGCAATGCG
GCGGCTGCATACGCTTGATCCGGCTACCTGCCCATTCGACCACCAAGCGAAACATCGCAT
CGAGCGAGCACGTACTCGGATGGAAGCCGGTCTTGTCGATCAGGATGATCTGGACGAAGA
GCATCAGGGGCTCGCGCCAGCCGAACTGTTCGCCAGGCTCAAGGCGAGCATGCCCGACGG
CGAGGATCTCGTCGTGACCCATGGCGATGCCTGCTTGCCGAATATCATGGTGGAAAATGG
CCGCTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTTCCC
CGTGCTTTACGGTATCGCCGCTCCCGATTCGCAGCGCATCGCCTTCTATCGCCTTCTTGA
CGAGTTCTTCTGAATTATTAACGCTTACAATTTCCTGATGCGGTATTTTCTCCTTACGCA
TCTGTGCGGTATTTCACACCGCATCAGGTGGCACTTTTCGGGGAAATGTGCGCGGAACCC
CTATTTGTTTATTTTTCTAAATACATTCAAATATGTATCCGCTCATGAGACAATAACCCT
GATAAATGCTTCAATAATAGCACGTGCTAAAACTTCATTTTTAATTTAAAAGGATCTAGG
TGAAGATCCTTTTTGATAATCTCATGACCAAAATCCCTTAACGTGAGTTTTCGTTCCACT
GAGCGTCAGACCCCGTAGAAAAGATCAAAGGATCTTCTTGAGATCCTTTTTTTCTGCGCG
TAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATA
CTGTTCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGATACCTAC
AGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTTACGGTTCCTGG
CCTTTTGCTGGCCTTTTGCTCACATGTTCTT
RNA coding sequence for CD45-BiTE-2 (HLHL)
SEQ ID NO: 18
ATGGACTGGACCTGGATCCTGTTCCTGGTGGCTGCCGCCACCAGGGTGCACAGCGACAAG
ACCCACACATGCCCTCCATGTCCAGCACCAGAGGCAGCCGGCGGACCTAGCGTGTTCCTG
TTTCCCCCTAAGCCAAAGGATACCCTGATGATCTCTCGGACCCCTGAGGTGACATGCGTG
GTGGTGGACGTGAGCCACGAGGCACCAGAGGTGAAGTTCAACTGGTACGTGGATGGCGTG
GAGGTGCACAATGCCAAGACAAAGCCTTGCGAGGAGCAGTACGGCTCCACCTATCGCTGC
GTGAGCGTGCTGACAGTGCTGCACCAGGACTGGCTGAACGGCAAGGAGTATAAGTGCGCC
GTGTCCAATAAGGCCCTGCCTGCCCCAATCGAGAAGACCATCTCTAAGGCCAAGGGCCAG
CCTAGGGAGCCACAGGTGTACACACTGCCACCCTCCAGAGAGGAGATGACCAAGAACCAG
GTGTCTCTGACATGTCTGGTGAAGGGCTTCTATCCCAGCGACATCGCCGTGGAGTGGGAG
TCCAATGGCCAGCCTGAGAACAATTACAAGACCACACCTCCAGTGCTGGACTCTGATGGC
AGCTTCTTTCTGTATTCTAAGCTGACCGTGGATAAGAGCCGGTGGCAGCAGGGCAACGTG
TTTAGCTGCTCCGTGATGCACGAGGCCCTGCACAATCACTACACACAGAAGTCTCTGAGC
CTGTCCCCAGGCAAGGGCGGCGGCGGCAGCGGCGGCGGCGGCAGCGGCGGCGGCGGCAGC
GGCGGCGGCGGCAGCGGCGGCGGCGGCAGCGGCGGCGGCGGCTCGGATAAAACCCACACA
TGCCCTCCCTGTCCAGCACCTGAGGCAGCCGGCGGACCAAGCGTGTTCCTGTTTCCACCC
AAGCCTAAAGACACCCTGATGATCAGCAGGACCCCCGAAGTCACCTGCGTGGTCGTGGAC
GTGTCCCACGAGGCACCTGAAGTCAAGTTCAACTGGTACGTGGACGGAGTCGAAGTCCAT
AACGCCAAGACAAACCCCTGTGAAGAGCAGTACGGCAGCACCTATAGATGCGTGAGCGTG
CTGACAGTGCTGCATCAGGATTGGCTGAATGGCAAGGAATACAAGTGCGCCGTGTCTAAT
AAGGCCCTGCCAGCCCCCATCGAGAAGACCATCTCCAAGGCAAAGGGACAGCCAAGGGAG
CCTCAGGTGTACACACTGCCTCCAAGCCGCGAAGAAATGACTAAAAACCAGGTGTCCCTG
ACCTGCCTGGTCAAAGGCTTCTATCCATCTGATATTGCCGTGGAGTGGGAGAGCAATGGC
CAGCCCGAAAATAATTACAAGACCACACCCCCTGTGCTGGACTCCGATGGCTCTTTCTTT
CTGTATTCCAAACTGACTGTGGATAAGTCTCGCTGGCAGCAGGGAAACGTGTTTTCTTGT
AGCGTGATGCATGAGGCCCTGCATAACCACTACACACAGAAGTCCCTGTCTCTGAGCCCT
GGCAAGGGCGGCGGCGGCAGCGGCGGCGGCGGCTCTCAGGTGCAGCTGGTGGAGAGCGGC
GGCGGCCTGGTGCAGCCTGGCGGCTCCCTGAAGCTGTCTTGCGCAGCAAGCGGCTTCGAC
TTTTCTAGGTACTGGATGAGCTGGGTGAGACAGGCACCAGGCAAGGGACTGGAGTGGATC
GGCGAGATCAACCCAACCAGCTCCACAATCAATTTCACCCCCTCCCTGAAGGACAAGGTG
TTTATCTCTCGGGATAACGCCAAGAATACCCTGTATCTGCAGATGTCCAAGGTGCGCTCT
GAGGACACAGCCCTGTACTATTGCGCCAGGGGCAACTACTATAGATACGGCGACGCCATG
GATTATTGGGGCCAGGGCACCTCCGTGACAGTGTCTAGCGGCGGCGGCGGCAGCGGGGGG
GGCGGCAGCGGCGGCGGCGGCTCCGATATCGTGCTGACACAGTCTCCAGCAAGCCTGGCC
GTGAGCCTGGGACAGAGGGCAACCATCTCCTGTAGGGCCTCCAAGTCTGTGAGCACATCC
GGCTACAGCTATCTGCACTGGTACCAGCAGAAGCCAGGACAGCCCCCTAAGCTGCTGATC
TATCTGGCAAGCAACCTGGAGTCCGGAGTGCCTGCAAGGTTCTCTGGAAGCGGATCCGGA
ACCGACTTTACACTGAATATCCACCCAGTGGAGGAGGAGGATGCCGCCACCTACTATTGT
CAGCACTCTAGGGAGCTGCCCTTCACCTTTGGCAGCGGCACAAAGCTGGAGATCAAGGGC
GGCGGCGGCTCGGAGGTGCAGCTGGTGGAGAGCGGCGGCGGCCTGGTGCAGCCAGGCGGC
AGCCTGAGGCTGTCCTGTGCAGCAAGCGGATACTCCTTCACCGGCTATACAATGAATTGG
GTGAGGCAGGCCCCTGGCAAGGGCCTGGAATGGGTGGCCCTGATCAACCCCTACAAGGGC
GTGTCCACCTATAATCAGAAGTTCAAGGACCGCTTTACCATCTCTGTGGATAAGAGCAAG
AACACAGCCTACCTGCAGATGAATAGCCTGAGAGCCGAGGACACAGCCGTGTACTATTGC
GCACGGAGCGGATACTATGGCGACTCCGATTGGTATTTTGACGTGTGGGGCCAGGGCACC
CTGGTGACAGTGAGCAGCGGCGGCGGCGGCAGCGGCGGCGGCGGCAGCGGCGGCGGCGGC
TCTGACATCCAGATGACCCAGTCCCCATCTAGCCTGTCTGCCAGCGTCGGCGACAGGGTG
ACCATCACATGTCGCGCCTCTCAGGATATCAGGAACTACCTGAATTGGTATCAGCAGAAG
CCCGGCAAGGCCCCTAAGCTGCTGATCTACTATACATCCAGGCTGGAGTCTGGAGTGCCA
AGCAGGTTCTCCGGATCTGGAAGCGGAACCGACTACACCCTGACAATCTCCTCTCTGCAG
CCCGAGGATTTCGCCACATACTATTGTCAGCAGGGCAATACCCTGCCTTGGACATTTGGC
CAGGGCACCAAGGTGGAGATCAAGAGCTCCTGATAA
9.4 HLHL + his
(SEQ ID NO: 19)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSQVQLQQLGAELARPGASVKMSCKASGYT
FTSYSIQWVKQRPGQGLEWIGYINPSSGYIKYNQHFRDRATLTADRSSSTAYMQLSSLTS
EDSAVYYCARGNSGSFDYWGQGTTLTVSSGGGGSGGGGSGGGGSDIVMTQAAPSVPVTPG
ESLSISCRSSKSLLHSSGITYLYWFLQRPGQSPQLLIYRMSNLASGVPDRFSGSGSGTAF
TLRISRVEAEDVGVYYCMQHLEYPFTFGGGTKLEIKGGGGSEVQLVESGGGLVQPGGSLR
LSCAASGYSFTGYTMNWVRQAPGKGLEWVALINPYKGVSTYNQKFKDRFTISVDKSKNTA
YLQMNSLRAEDTAVYYCARSGYYGDSDWYFDVWGQGTLVTVSSGGGGSGGGGSGGGGSDI
QMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKSSHHHHHH
9.4 HLHL
(SEQ ID NO: 20)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSQVQLQQLGAELARPGASVKMSCKASGYT
FTSYSIQWVKQRPGQGLEWIGYINPSSGYIKYNQHFRDRATLTADRSSSTAYMQLSSLTS
EDSAVYYCARGNSGSFDYWGQGTTLTVSSGGGGSGGGGSGGGGSDIVMTQAAPSVPVTPG
ESLSISCRSSKSLLHSSGITYLYWFLQRPGQSPQLLIYRMSNLASGVPDRFSGSGSGTAF
TLRISRVEAEDVGVYYCMQHLEYPFTFGGGTKLEIKGGGGSEVQLVESGGGLVQPGGSLR
LSCAASGYSFTGYTMNWVRQAPGKGLEWVALINPYKGVSTYNQKFKDRFTISVDKSKNTA
YLQMNSLRAEDTAVYYCARSGYYGDSDWYFDVWGQGTLVTVSSGGGGSGGGGSGGGGSDI
QMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKSS
9.4 LHHL + his
(SEQ ID NO: 21)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSDIVMTQAAPSVPVTPGESLSISCRSSKS
LLHSSGITYLYWFLQRPGQSPQLLIYRMSNLASGVPDRFSGSGSGTAFTLRISRVEAEDV
GVYYCMQHLEYPFTFGGGTKLEIKGGGGSGGGGSGGGGSQVQLQQLGAELARPGASVKMS
CKASGYTFTSYSIQWVKQRPGQGLEWIGYINPSSGYIKYNQHFRDRATLTADRSSSTAYM
QLSSLTSEDSAVYYCARGNSGSFDYWGQGTTLTVSSGGGGSEVQLVESGGGLVQPGGSLR
LSCAASGYSFTGYTMNWVRQAPGKGLEWVALINPYKGVSTYNQKFKDRFTISVDKSKNTA
YLQMNSLRAEDTAVYYCARSGYYGDSDWYFDVWGQGTLVTVSSGGGGSGGGGSGGGGSDI
QMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKSSHHHHHH
9.4 LHHL
(SEQ ID NO: 22)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSDIVMTQAAPSVPVTPGESLSISCRSSKS
LLHSSGITYLYWFLQRPGQSPQLLIYRMSNLASGVPDRFSGSGSGTAFTLRISRVEAEDV
GVYYCMQHLEYPFTFGGGTKLEIKGGGGSGGGGSGGGGSQVQLQQLGAELARPGASVKMS
CKASGYTFTSYSIQWVKQRPGQGLEWIGYINPSSGYIKYNQHFRDRATLTADRSSSTAYM
QLSSLTSEDSAVYYCARGNSGSFDYWGQGTTLTVSSGGGGSEVQLVESGGGLVQPGGSLR
LSCAASGYSFTGYTMNWVRQAPGKGLEWVALINPYKGVSTYNQKFKDRFTISVDKSKNTA
YLQMNSLRAEDTAVYYCARSGYYGDSDWYFDVWGQGTLVTVSSGGGGSGGGGSGGGGSDI
QMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKSS
Gap8.3 HLHL + his
(SEQ ID NO: 23)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSEVQLQQSGPELVKTGASVKISCKASGYS
FTGYFIHWVKQSHGKSLEWIGYISCYNGATSYNQKFKGKATFTVDTSSSTAYMQFNSVTS
EDSAVYYCVRNYYGNLDAMDYWGQGTSVTVSSGGGGSGGGGSGGGGSDIVMTQSHKFMST
SVGDRVSITCKASQDVSTAVAWYQQKPGQSPKILIYSASYRYTGVPDRFTGSGSGTDFTF
TISSVQAEDLAVYYCQQHYSTPRTFGGGTKLEIKGGGGSEVQLVESGGGLVQPGGSLRLS
CAASGYSFTGYTMNWVRQAPGKGLEWVALINPYKGVSTYNQKFKDRFTISVDKSKNTAYL
QMNSLRAEDTAVYYCARSGYYGDSDWYFDVWGQGTLVTVSSGGGGSGGGGSGGGGSDIQM
TQSPSSLSASVGDRVTITCRASQDIRNYLNWYQQKPGKAPKLLIYYTSRLESGVPSRFSG
SGSGTDYTLTISSLQPEDFATYYCQQGNTLPWTFGQGTKVEIKSSHHHHHH
Gap8.3 HLHL
(SEQ ID NO: 24)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSEVQLQQSGPELVKTGASVKISCKASGYS
FTGYFIHWVKQSHGKSLEWIGYISCYNGATSYNQKFKGKATFTVDTSSSTAYMQFNSVTS
EDSAVYYCVRNYYGNLDAMDYWGQGTSVTVSSGGGGSGGGGSGGGGSDIVMTQSHKFMST
SVGDRVSITCKASQDVSTAVAWYQQKPGQSPKILIYSASYRYTGVPDRFTGSGSGTDFTF
TISSVQAEDLAVYYCQQHYSTPRTFGGGTKLEIKGGGGSEVQLVESGGGLVQPGGSLRLS
CAASGYSFTGYTMNWVRQAPGKGLEWVALINPYKGVSTYNQKFKDRFTISVDKSKNTAYL
QMNSLRAEDTAVYYCARSGYYGDSDWYFDVWGQGTLVTVSSGGGGSGGGGSGGGGSDIQM
TQSPSSLSASVGDRVTITCRASQDIRNYLNWYQQKPGKAPKLLIYYTSRLESGVPSRFSG
SGSGTDYTLTISSLQPEDFATYYCQQGNTLPWTFGQGTKVEIKSS
Gap8.3 LHHL + his
(SEQ ID NO: 25)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSDIVMTQSHKFMSTSVGDRVSITCKASQD
VSTAVAWYQQKPGQSPKILIYSASYRYTGVPDRFTGSGSGTDFTFTISSVQAEDLAVYYC
QQHYSTPRTFGGGTKLEIKGGGGSGGGGSGGGGSEVQLQQSGPELVKTGASVKISCKASG
YSFTGYFIHWVKQSHGKSLEWIGYISCYNGATSYNQKFKGKATFTVDTSSSTAYMQFNSV
TSEDSAVYYCVRNYYGNLDAMDYWGQGTSVTVSSGGGGSEVQLVESGGGLVQPGGSLRLS
CAASGYSFTGYTMNWVRQAPGKGLEWVALINPYKGVSTYNQKFKDRFTISVDKSKNTAYL
QMNSLRAEDTAVYYCARSGYYGDSDWYFDVWGQGTLVTVSSGGGGSGGGGSGGGGSDIQM
TQSPSSLSASVGDRVTITCRASQDIRNYLNWYQQKPGKAPKLLIYYTSRLESGVPSRFSG
SGSGTDYTLTISSLQPEDFATYYCQQGNTLPWTFGQGTKVEIKSSHHHHHH
Gap8.3 LHHL
(SEQ ID NO: 26)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSDIVMTQSHKFMSTSVGDRVSITCKASQD
VSTAVAWYQQKPGQSPKILIYSASYRYTGVPDRFTGSGSGTDFTFTISSVQAEDLAVYYC
QQHYSTPRTFGGGTKLEIKGGGGSGGGGSGGGGSEVQLQQSGPELVKTGASVKISCKASG
YSFTGYFIHWVKQSHGKSLEWIGYISCYNGATSYNQKFKGKATFTVDTSSSTAYMQFNSV
TSEDSAVYYCVRNYYGNLDAMDYWGQGTSVTVSSGGGGSEVQLVESGGGLVQPGGSLRLS
CAASGYSFTGYTMNWVRQAPGKGLEWVALINPYKGVSTYNQKFKDRFTISVDKSKNTAYL
QMNSLRAEDTAVYYCARSGYYGDSDWYFDVWGQGTLVTVSSGGGGSGGGGSGGGGSDIQM
TQSPSSLSASVGDRVTITCRASQDIRNYLNWYQQKPGKAPKLLIYYTSRLESGVPSRFSG
SGSGTDYTLTISSLQPEDFATYYCQQGNTLPWTFGQGTKVEIKSS
2:1 CD45xCD3
(SEQ ID NO: 27)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSQVQLVESGGGLVQPGGSLKLSCAASGFD
FSRYWMSWVRQAPGKGLEWIGEINPTSSTINFTPSIKDKVFTISRDNLAKKTLYLQMSKV
RSEDTALYYCARGNYYRYGDAMDYWGQGTSVTVSSGGGGSGGGGSGGGGSDIVLTQSPAS
LAVSLGQRATISCRASKSVSTSGYSYLHWYQQKPGQPPKLLIYLASNLESGVPARFSGSG
SGTDFTLNIHPVEEEDAATYYCQHSRELPFTFGSGTKLEIKGGGGSEVQLVESGGGLVQP
GGSLRLSCAASGYSFTGYTMNWVRQAPGKGLEWVALINPYKGVSTYNQKFKDRFTISVDK
SKNTAYLQMNSLRAEDTAVYYCARSGYYGDSDWYFDVWGQSTLVTVSSGGGGSGGGGSGG
GGSDIQMTQSPSSLSASVGDRVTITCRASQDIRNYLNWYQQKPGKAPKLLIYYTSRLESG
VPSRFSGSGSGTDYTLTISSLOPEDFATYYCQQGNTLPWTFGQGTKVEIKSSGGGGSQVQ
LVESGGGLVQPGGSLKLSCAASGFDFSRYWMSWVRQAPGKGLEWIGEINPTSSTINFTPS
LKDKVFISRDNAKNTLYLQMSKVRSEDTALYYCARGNYYRYGDAMDYWGQGTSCTVSSGG
GGGGGGGGGGSDIVLTQSPASLAVSLGQRATISCRASKSVSTSGYSYLHWYQQKPGQPPK
LLIYLASNLESGVPARFSGSGSGTDFTLNIHPVEEEDAATYYCQHSRELPFTFGSGTKLE
IK
2:1 CD45xCD3 (with His)
(SEQ ID NO: 28)
MDWTWILFLVAAATRVHSDKTHTCPPCPAPEAAGGPSVFLFPPKPKDTLMISRTPEVTCV
VVDVSHEAPEVKFNWYVDGVEVHNAKTKPCEEQYGSTYRCVSVLTVLHQDWLNGKEYKCA
VSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLS
LSPGKGGGGSGGGGSGGGGSGGGGSGGGGSGGGGSDKTHTCPPCPAPEAAGGPSVFLFPP
KPKDTLMISRTPEVTCVVVDVSHEAPEVKFNWYVDGVEVHNAKTKPCEEQYGSTYRCVSV
LTVLHQDWLNGKEYKCAVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSC
SVMHEALHNHYTQKSLSLSPGKGGGGSGGGGSQVQLVESGGGLVQPGGSLKLSCAASGFD
FSRYWMSWVRQAPGKGLEWIGEINPTSSTINFTPSIKDKVFTISRDNLAKKTLYLQMSKV
RSEDTALYYCARGNYYRYGDAMDYWGQGTSVTVSSGGGGGGGGSGGGGSDIVLTQSPASL
AVSLGQRATISCRASKSVSTSGYSYLHWYQQKPGQPPKLLIYLASNLESGVPARFSGSGS
GTDFTLNIHPVEEEDAATYYCQHSRELPFTFGSGTKLEIKGGGGSEVQLVESGGGLVQPG
GSLRLSCAASGYSFTGYTMNWVRQAPGKGLEWVALINPYKGVSTYNQKFKDRFTISVDKS
KNTAYLQMNSLRAEDTAVYYCARSGYYGDSDWYFDVWGQSTLVTVSSGGGGSGGGGSGGG
GSDIQMTQSPSSLSASVGDRVTITCRASQDIRNYLNWYQQKPGKAPKLLIYYTSRLESGV
PSRFSGSGSGTDYTLTISSLQPEDFATYYCQQGNTLPWTFGQGTKVEIKSSGGGGSQVQL
VESGGGLVQPGGSLKLSCAASGFDFSRYWMSWVRQAPGKGLEWIGEINPTSSTINFTPSL
KDKVFISRDNAKNTLYLQMSKVRSEDTALYYCARGNYYRYGDAMDYWGQGTSCTVSSGGG
GSGGGGSGGGGSDIVLTQSPASLAVSLGQRATISCRASKSVSTSGYSYLHWYQQKPGQPP
KLLIYLASNLESGVPARFSGSGSGTDFTLNIHPVEEEDAATYYCQHSRELPFTFGSGTKL
EIKHHHHHH

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A synthetic multi-specific immune cell engager (ICE), wherein the synthetic multi-specific immune cell engager comprises at least one CD45 antigen binding domain, and at least one immune cell engaging domain.

2. The synthetic multi-specific ICE of claim 1, wherein the synthetic multi-specific immune cell engager comprises at least two CD45 antigen binding domains, and at least one immune cell engaging domain.

3. The synthetic multi-specific ICE of claim 1 or claim 2, comprising at least one antigen binding domain comprising:
a) a variable heavy chain amino acid sequence having at least about 90% identity over the entire length of the amino acid sequence to SEQ ID NO:1 comprising at least the CDR sequences of SEQ ID NO:1; or a fragment of SEQ ID NO:1 comprising at least the CDR sequences of SEQ ID NO: 1; and
b) a variable light chain amino acid sequence an amino acid sequence having at least about 90% identity over the entire length of the amino acid sequence to SEQ ID NO:2 comprising at least the CDR sequences of SEQ ID NO:2; or a fragment of SEQ ID NO:2 comprising at least the CDR sequences of SEQ ID NO:2.

4. The synthetic multi-specific ICE of any one of claims 1-3, comprising at least one antigen binding domain comprising an amino acid sequence having at least about 90% identity over the entire length of the amino acid sequence to SEQ ID NO:5 or SEQ ID NO:7, wherein the amino acid sequence comprising at least the CDR sequences of SEQ ID NO:5 or SEQ ID NO:7; or a fragment of SEQ ID NO:5 or SEQ ID NO:7 comprising at least the CDR sequences of SEQ ID NO:5 or SEQ ID NO:7.

5. The synthetic multi-specific ICE of claim 1, wherein the immune cell is a T cell or a naturally killer (NK) cell.

6. The synthetic multi-specific ICE of claim 1, wherein the immune cell engaging domain targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95.

7. The synthetic multi-specific ICE of claim 6, wherein the immune cell engaging domain targets CD3.

8. The synthetic multi-specific ICE of claim 7 comprising an antigen binding domain comprising
a) a variable heavy chain amino acid sequence having at least about 90% identity over the entire length of the amino acid sequence to SEQ ID NO:3 comprising at least the CDR sequences of SEQ ID NO:3; or a fragment of SEQ ID NO:3 comprising at least the CDR sequences of SEQ ID NO:3; and
b) a variable light chain amino acid sequence an amino acid sequence having at least about 90% identity over the entire length of the amino acid sequence to SEQ ID NO:4 comprising at least the CDR sequences of SEQ ID NO:4; or a fragment of SEQ ID NO:4 comprising at least the CDR sequences of SEQ ID NO:4.

9. The synthetic multi-specific ICE of any one of claims 5-8 comprising an amino acid sequence having at least about 90% identity over the entire length of the amino acid sequence to SEQ ID NO:9, wherein the amino acid sequence comprising at least the CDR sequences of SEQ ID NO:9; or a fragment of SEQ ID NO:9 comprising at least the CDR sequences of SEQ ID NO: 9.

10. The synthetic multi-specific ICE of claim 1 comprising an amino acid sequence having at least about 90% identity over the entire length of the amino acid sequence to SEQ ID NO: 11, SEQ ID NO: 12, SEQ ID NO: 13, SEQ ID NO: 14, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28, wherein the amino acid sequence comprising at least the CDR sequences of SEQ ID NO:11, SEQ ID NO:12, SEQ ID NO:13, SEQ ID NO:14, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28; or a fragment of SEQ ID NO: 11, SEQ ID NO: 12, SEQ ID NO:13, SEQ ID NO:14, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 comprising at least the CDR sequences of SEQ ID NO:11, SEQ ID NO: 12, SEQ ID NO: 13, SEQ ID NO:14, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28.

11. A nucleic acid molecule encoding a synthetic multi-specific ICE of any one of claims 1-10.

12. The nucleic acid molecule of claim 11, wherein the nucleic acid molecule is selected from the group consisting of an RNA molecule and a DNA molecule.

13. The nucleic acid molecule of claim 11 or claim 12, comprising a nucleotide sequence selected from the group consisting of:
a) a nucleotide sequence having at least about 90% identity over an entire length of the nucleic acid sequence to SEQ ID NO:6, SEQ ID NO:8, or SEQ ID NO:10;
b) a fragment of a nucleotide sequence having at least about 90% identity over at least 65% of the nucleic acid sequence to SEQ ID NO:6, SEQ ID NO:8, or SEQ ID NO: 10;
c) a nucleotide sequence of SEQ ID NO:6, SEQ ID NO:8, or SEQ ID NO:10; and
d) a fragment of a nucleotide sequence comprising at least 65% of a nucleotide sequence of SEQ ID NO:6, SEQ ID NO:8, or SEQ ID NO:10.

14. The nucleic acid molecule of claim 13, wherein the nucleic acid molecule comprises a nucleotide sequence selected from SEQ ID NO:15, SEQ ID NO: 16, SEQ ID NO: 17 or SEQ ID NO:18, or a fragment or variant thereof.

15. The nucleic acid molecule of any one of claims 11-14, wherein the nucleic acid molecule comprises an expression vector.

16. A composition comprising the multi-specific ICE of any one of claims 1-10 or the nucleic acid molecule of any one of claims 11-15.

17. The composition of claim 16, further comprising a pharmaceutically acceptable excipient.

18. The composition of claim 16, further comprising at least one immune checkpoint inhibitor.

19. The composition of claim 16, further comprising at least one nucleic acid molecule encoding at least one immune checkpoint inhibitor.

20. The composition of claim 18 or 19, wherein the immune checkpoint inhibitor is selected from the group consisting of an inhibitor of PD-1, an inhibitor of PD-L-1, an inhibitor of cytotoxic T-lymphocyte antigen-4 (CTLA-4), an inhibitor of mucin-domain containing-3 (TIM-3), and an inhibitor of Lymphocyte Activating 3 (LAG3).

21. The composition of claim 16, wherein the composition comprises a lipid nanoparticle comprising the multi-specific ICE of any one of claims 1-10 or the nucleic acid molecule of any one of claims 11-15.

22. A method of preventing or treating CD45 expressing cancer in a subject, the method comprising administering to the subject multi-specific ICE of any one of claims 1-10, the nucleic acid molecule of any one of claims 11-15 or the composition of any one of claims 12-21.

23. The method of claim 22, wherein the CD45 expressing cancer is a blood cancer.

24. The method of claim 22, wherein the blood cancer is selected from the group consisting of leukemia, myeloma, and lymphoma.

25. The method of claim 22, wherein the blood cancer is acute myeloid leukemia (AML).

26. A CD45-specific chimeric antigen receptor (CAR) molecule.

27. The CAR molecule of claim 26, wherein the CAR comprises an CD45 specific binding arm comprising a nucleotide sequence as set forth in SEQ ID NO:6 or SEQ ID NO:8.

28. A composition comprising an CD45-specific chimeric antigen receptor (CAR) molecule of any one of claims 26-27.

29. The composition of claim 28, wherein the composition comprises a cell expressing the CD45-specific chimeric antigen receptor (CAR) molecule.

30. The composition of claim 28, wherein the cell is an engineered T cell.

31. A method of preventing or treating a disease or disorder in a subject in need thereof, the method comprising administering to the subject
a) a multi-specific ICE wherein the synthetic multi-specific immune cell engager comprises at least one least one antigen binding domain, and at least one immune cell engaging domain, wherein the antigen binding domain is specific for a disease-associated antigen, and
b) an engineered cell, wherein the engineered cell comprises a modification of at least one epitope that is bound by the multi-specific ICE.

32. The method of claim 31, wherein the multi-specific antibody targets one or more of CD45, CD3, CD2, CD5, CD7, CD19, CD20, CD22, CD33, CCR5, CCR8, and BCMA.

33. The method of claim 31, wherein the engineered cell comprises a modification in one or more of CD45, CD3, CD2, CD5, CD7, CD19, CD20, CD22, CD33, CCR5, CCR8, and BCMA to reduce binding of the multi-specific antibody to the engineered cell.

34. The method of claim 31, wherein the engineered cell is selected from the group consisting of hematopoietic stem cells, T cells, B cells, NK cells, NK-T cells, neutrophils, macrophages, monocytes, dendritic cells, or any combination thereof.

35. The method of claim 31, wherein the engineered cell is selected from the group consisting of autologous cells and allogeneic cells with respect to the subject.

36. The method of claim 31, wherein the engineered cell is epitope edited ex vivo prior to administration.

37. The method of claim 31, wherein the disease or disorder is cancer, an autoimmune disease, or an infectious disease.

38. The method of claim 31, wherein the disease or disorder is AML.


SEQUENCE LISTING (28 sequences)

SEQ ID NO: 1 - protein, 121 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSS

SEQ ID NO: 2 - protein, 111 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 3 - protein, 122 aa, synthetic construct [sequence omitted]

SEQ ID NO: 4 - protein, 109 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 5 - protein, 247 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 6 - DNA, 741 bp, synthetic construct
  source 1..741: mol_type = other DNA
caggtgcagctggtggagagcggcggcggcctggtgcagcctggcggctccctgaagctgtcttgcgcagcaagcggcttcgacttttctaggtactggatgagctgggtgagacaggcaccaggcaagggactggagtggatcggcgagatcaacccaaccagctccacaatcaatttcaccccctccctgaaggacaaggtgtttatctctcgggataacgccaagaataccctgtatctgcagatgtccaaggtgcgctctgaggacacagccctgtactattgcgccaggggcaactactatagatacggcgacgccatggattattggggccagggcacctccgtgacagtgtctagcggcggcggcggcagcggcggcggcggcagcggcggcggcggctccgatatcgtgctgacacagtctccagcaagcctggccgtgagcctgggacagagggcaaccatctcctgtagggcctccaagtctgtgagcacatccggctacagctatctgcactggtaccagcagaagccaggacagccccctaagctgctgatctatctggcaagcaacctggagtccggagtgcctgcaaggttctctggaagcggatccggaaccgactttacactgaatatccacccagtggaggaggaggatgccgccacctactattgtcagcactctagggagctgcccttcacctttggcagcggcacaaagctggagatcaag

SEQ ID NO: 7 - protein, 498 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 8 - DNA, 741 bp, synthetic construct
  source 1..741: mol_type = other DNA
gacatcgtgctgacccagtctccagcaagcctggccgtgtccctgggacagagggcaaccatctcttgcagggcctccaagtctgtgagcacatccggctactcctatctgcactggtaccagcagaagccaggacagccccctaagctgctgatctatctggcctccaacctggagtctggagtgcctgcaaggttctctggaagcggatccggaaccgactttacactgaatatccacccagtggaggaggaggatgccgccacatactattgccagcactctagggagctgcccttcacctttggcagcggcacaaagctggagatcaagggcggcggcggcagcggcggcggcggcagcggcggcggcggctcccaggtgcagctggtggagagcggcggcggcctggtgcagcctggcggcagcctgaagctgtcctgtgcagcatctggcttcgacttttctaggtactggatgagctgggtgagacaggcaccaggcaagggactggagtggatcggcgagatcaacccaaccagctccacaatcaatttcacccccagcctgaaggacaaggtgtttatctcccgggataacgccaagaataccctgtatctgcagatgagcaaggtgcgctccgaggatacagccctgtactattgtgccaggggcaactactatagatacggcgacgccatggattattggggccagggcacctccgtgacagtgtctagc

SEQ ID NO: 9 - protein, 246 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 10 - DNA, 738 bp, synthetic construct
  source 1..738: mol_type = other DNA
gaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcc

SEQ ID NO: 11 - protein, 1010 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 12 - protein, 1016 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 13 - protein, 1010 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 14 - protein, 1016 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 15 - DNA, 6023 bp, synthetic construct
  source 1..6023: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccgccaccatggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgatgcacgaggccctgcacaatcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgatgcatgaggccctgcataaccactacacacagaagtccctgtctctgagccctggcaagggcggcggcggcagcggcggcggcggctctgacatcgtgctgacccagtctccagcaagcctggccgtgtccctgggacagagggcaaccatctcttgcagggcctccaagtctgtgagcacatccggctactcctatctgcactggtaccagcagaagccaggacagccccctaagctgctgatctatctggcctccaacctggagtctggagtgcctgcaaggttctctggaagcggatccggaaccgactttacactgaatatccacccagtggaggaggaggatgccgccacatactattgccagcactctagggagctgcccttcacctttggcagcggcacaaagctggagatcaagggcggcggcggcagcggcggcggcggcagcggcggcggcggctcccaggtgcagctggtggagagcggcggcggcctggtgcagcctggcggcagcctgaagctgtcctgtgcagcatctggcttcgacttttctaggtactggatgagctgggtgagacaggcaccaggcaagggactggagtggatcggcgagatcaacccaaccagctccacaatcaatttcacccccagcctgaaggacaaggtgtttatctcccgggataacgccaagaataccctgtatctgcagatgagcaaggtgcgctccgaggatacagccctgtactattgtgccaggggcaactactatagatacggcgacgccatggattattggggccagggcacctccgtgacagtgtctagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 16 - DNA, 3036 bp, synthetic construct
  source 1..3036: mol_type = other DNA
atggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgatgcacgaggccctgcacaatcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgatgcatgaggccctgcataaccactacacacagaagtccctgtctctgagccctggcaagggcggcggcggcagcggcggcggcggctctgacatcgtgctgacccagtctccagcaagcctggccgtgtccctgggacagagggcaaccatctcttgcagggcctccaagtctgtgagcacatccggctactcctatctgcactggtaccagcagaagccaggacagccccctaagctgctgatctatctggcctccaacctggagtctggagtgcctgcaaggttctctggaagcggatccggaaccgactttacactgaatatccacccagtggaggaggaggatgccgccacatactattgccagcactctagggagctgcccttcacctttggcagcggcacaaagctggagatcaagggcggcggcggcagcggcggcggcggcagcggcggcggcggctcccaggtgcagctggtggagagcggcggcggcctggtgcagcctggcggcagcctgaagctgtcctgtgcagcatctggcttcgacttttctaggtactggatgagctgggtgagacaggcaccaggcaagggactggagtggatcggcgagatcaacccaaccagctccacaatcaatttcacccccagcctgaaggacaaggtgtttatctcccgggataacgccaagaataccctgtatctgcagatgagcaaggtgcgctccgaggatacagccctgtactattgtgccaggggcaactactatagatacggcgacgccatggattattggggccagggcacctccgtgacagtgtctagcggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataa

SEQ ID NO: 17 - DNA, 6023 bp, synthetic construct
  source 1..6023: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccgccaccatggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgatgcacgaggccctgcacaatcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgatgcatgaggccctgcataaccactacacacagaagtccctgtctctgagccctggcaagggcggcggcggcagcggcggcggcggctctcaggtgcagctggtggagagcggcggcggcctggtgcagcctggcggctccctgaagctgtcttgcgcagcaagcggcttcgacttttctaggtactggatgagctgggtgagacaggcaccaggcaagggactggagtggatcggcgagatcaacccaaccagctccacaatcaatttcaccccctccctgaaggacaaggtgtttatctctcgggataacgccaagaataccctgtatctgcagatgtccaaggtgcgctctgaggacacagccctgtactattgcgccaggggcaactactatagatacggcgacgccatggattattggggccagggcacctccgtgacagtgtctagcggcggcggcggcagcggcggcggcggcagcggcggcggcggctccgatatcgtgctgacacagtctccagcaagcctggccgtgagcctgggacagagggcaaccatctcctgtagggcctccaagtctgtgagcacatccggctacagctatctgcactggtaccagcagaagccaggacagccccctaagctgctgatctatctggcaagcaacctggagtccggagtgcctgcaaggttctctggaagcggatccggaaccgactttacactgaatatccacccagtggaggaggaggatgccgccacctactattgtcagcactctagggagctgcccttcacctttggcagcggcacaaagctggagatcaagggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 18 - DNA, 3036 bp, synthetic construct
  source 1..3036: mol_type = other DNA
atggactggacctggatcctgttcctggtggctgccgccaccagggtgcacagcgacaagacccacacatgccctccatgtccagcaccagaggcagccggcggacctagcgtgttcctgtttccccctaagccaaaggataccctgatgatctctcggacccctgaggtgacatgcgtggtggtggacgtgagccacgaggcaccagaggtgaagttcaactggtacgtggatggcgtggaggtgcacaatgccaagacaaagccttgcgaggagcagtacggctccacctatcgctgcgtgagcgtgctgacagtgctgcaccaggactggctgaacggcaaggagtataagtgcgccgtgtccaataaggccctgcctgccccaatcgagaagaccatctctaaggccaagggccagcctagggagccacaggtgtacacactgccaccctccagagaggagatgaccaagaaccaggtgtctctgacatgtctggtgaagggcttctatcccagcgacatcgccgtggagtgggagtccaatggccagcctgagaacaattacaagaccacacctccagtgctggactctgatggcagcttctttctgtattctaagctgaccgtggataagagccggtggcagcagggcaacgtgtttagctgctccgtgatgcacgaggccctgcacaatcactacacacagaagtctctgagcctgtccccaggcaagggcggcggcggcagcggcggcggcggcagcggcggcggcggcagcggcggcggcggcagcggcggcggcggcagcggcggcggcggctcggataaaacccacacatgccctccctgtccagcacctgaggcagccggcggaccaagcgtgttcctgtttccacccaagcctaaagacaccctgatgatcagcaggacccccgaagtcacctgcgtggtcgtggacgtgtcccacgaggcacctgaagtcaagttcaactggtacgtggacggagtcgaagtccataacgccaagacaaagccctgtgaagagcagtacggcagcacctatagatgcgtgagcgtgctgacagtgctgcatcaggattggctgaatggcaaggaatacaagtgcgccgtgtctaataaggccctgccagcccccatcgagaagaccatctccaaggcaaagggacagccaagggagcctcaggtgtacacactgcctccaagccgcgaagaaatgactaaaaaccaggtgtccctgacctgcctggtcaaaggcttctatccatctgatattgccgtggagtgggagagcaatggccagcccgaaaataattacaagaccacaccccctgtgctggactccgatggctctttctttctgtattccaaactgactgtggataagtctcgctggcagcagggaaacgtgttttcttgtagcgtgatgcatgaggccctgcataaccactacacacagaagtccctgtctctgagccctggcaagggcggcggcggcagcggcggcggcggctctcaggtgcagctggtggagagcggcggcggcctggtgcagcctggcggctccctgaagctgtcttgcgcagcaagcggcttcgacttttctaggtactggatgagctgggtgagacaggcaccaggcaagggactggagtggatcggcgagatcaacccaaccagctccacaatcaatttcaccccctccctgaaggacaaggtgtttatctctcgggataacgccaagaataccctgtatctgcagatgtccaaggtgcgctctgaggacacagccctgtactattgcgccaggggcaactactatagatacggcgacgccatggattattggggccagggcacctccgtgacagtgtctagcggcggcggcggcagcggcggcggcggcagcggcggcggcggctccgatatcgtgctgacacagtctccagcaagcctggccgtgagcctgggacagagggcaaccatctcctgtagggcctccaagtctgtgagcacatccggctacagctatctgcactggtaccagcagaagccaggacagccccctaagctgctgatctatctggcaagcaacctggagtccggagtgcctgcaaggttctctggaagcggatccggaaccgactttacactgaatatccacccagtggaggaggaggatgccgccacctactattgtcagcactctagggagctgcccttcacctttggcagcggcacaaagctggagatcaagggcggcggcggctcggaggtgcagctggtggagagcggcggcggcctggtgcagccaggcggcagcctgaggctgtcctgtgcagcaagcggatactccttcaccggctatacaatgaattgggtgaggcaggcccctggcaagggcctggaatgggtggccctgatcaacccctacaagggcgtgtccacctataatcagaagttcaaggaccgctttaccatctctgtggataagagcaagaacacagcctacctgcagatgaatagcctgagagccgaggacacagccgtgtactattgcgcacggagcggatactatggcgactccgattggtattttgacgtgtggggccagggcaccctggtgacagtgagcagcggcggcggcggcagcggcggcggcggcagcggcggcggcggctctgacatccagatgacccagtccccatctagcctgtctgccagcgtcggcgacagggtgaccatcacatgtcgcgcctctcaggatatcaggaactacctgaattggtatcagcagaagcccggcaaggcccctaagctgctgatctactatacatccaggctggagtctggagtgccaagcaggttctccggatctggaagcggaaccgactacaccctgacaatctcctctctgcagcccgaggatttcgccacatactattgtcagcagggcaataccctgccttggacatttggccagggcaccaaggtggagatcaagagctcctgataa

SEQ ID NO: 19 - protein, 1013 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSGGGGSGGGGSGGGGSDIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 20 - protein, 1007 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSGGGGSGGGGSGGGGSDIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 21 - protein, 1013 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKGGGGSGGGGSGGGGSQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 22 - protein, 1007 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKGGGGSGGGGSGGGGSQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 23 - protein, 1011 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSGGGGSGGGGSGGGGSDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 24 - protein, 1005 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSGGGGSGGGGSGGGGSDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 25 - protein, 1011 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKGGGGSGGGGSGGGGSEVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSHHHHHH

SEQ ID NO: 26 - protein, 1005 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKGGGGSGGGGSGGGGSEVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 27 - protein, 1262 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 28 - protein, 1268 aa, synthetic construct
MDWTWILFLVAAATRVHSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPEAAGGPSVFLFPPKPKDTLMISRTPEVTCVVVDVSHEAPEVKFNWYVDGVEVHNAKTKPCEEQYGSTYRCVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKHHHHHH